(19) United States
(12) Patent Application Publication
(10) Pub. No.: US 2026/0199283 A1
(43) Pub. Date: Jul. 16, 2026

(54) METHODS AND COMPOUNDS FOR TREATMENT OF NEUROPATHIC PAIN

(71) Applicant: Algiax Pharmaceuticals GmbH, Erkrath (DE)

(72) Inventors: Birgit HASSE, Velbert (DE); Guido KOOPMANS, Sittard (NL)

(73) Assignee: Algiax Pharmaceuticals GmbH, Erkrath (DE)

(21) Appl. No.: 19/429,888

(22) Filed: Dec. 22, 2025

Related U.S. Application Data
(60) Provisional application No. 63/744,659, filed on Jan. 13, 2025.

(51) Int. Cl. A61K 31/277; A61P 25/04

(52) U.S. Cl. CPC A61K 31/277; A61P 25/04

(57) ABSTRACT

The present disclosure relates a method of treating neuropathic pain in human patients comprising a treatment cycle comprising:
administering a compound according to formula (I), or an isomer thereof, or a tautomer thereof, or an isotope thereof, over a period T¹; formula (I) having the structure of:

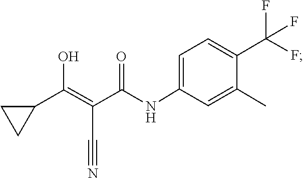 [SMILES: Cc1cc(NC(=O)/C(C#N)=C(\O)C2CC2)ccc1C(F)(F)F]

discontinuing the administration of the compound according to formula (I) for a period T² that immediately follows T¹;
obtaining pain reduction over the total sum of the periods T¹ and T² of the treatment cycle. The present disclosure also relates a compound according to formula (I) for use in treatment of neuropathic pain. The present disclosure also relates use of a compound according to formula (I) for preparing a pharmaceutical composition for treating a human patient suffering from neuropathic pain.

### CROSS-REFERENCE TO RELATED APPLICATION

The present application claims the benefit of priority of United States Provisional Application No. U.S. 63/744,659, filed Jan. 13, 2025, the content of which is hereby incorporated by reference in its entirety for all purposes.

### FIELD OF THE INVENTION

The present invention relates to methods of treatment, compounds for use in treatment and use of compounds according to formula (I) for preparing pharmaceutical compositions for treating neuropathic pain.

### BACKGROUND OF THE INVENTION

Neuropathic pain typically develops when peripheral or central nerves are damaged through surgery, disease or injury and is a major factor causing impaired quality of life in millions of people worldwide. The mechanisms involved in pain of neuropathic origin are complex and involve among other the diminished loss of GABAergic inhibition in the dorsal root ganglia (DRG) and the spinal dorsal horn. Many of the presently used medications for treating neuropathic pain must be taken on a daily basis and involve side effects that can disrupt a patient's state of wellbeing and complicate a return to quality. In the case of opiates, the side effects can also involve the threat of dependency or addiction to medication that can also be life threatening when abused. Many drugs that have been repurposed for treatment of neuropathic pain such as gabapentin or pregabalin can also have central nervous system side effect profiles that are disruptive such as dizziness or lethargy and can prevent patients from returning to activities that are necessary for work or directly affect the patients quality of life.

The present invention advantageously permits a significant reduction of neuropathic pain in patients suffering therefrom while drastically reducing the intake of medication and side effect profile of the neuropathic pain treatment. Surprisingly, the present invention permits long periods of neuropathic pain reduction that persist for significant periods of time after discontinuation of medication and after nearly all of the medication has been eliminated from the patient's body. Furthermore, the present treatment is peripherally acting and does not significantly penetrate the central nervous system, thus avoiding many of the CNS associated side effects of other medications. Unexpectedly, the present invention provides a treatment that eschews many of the complications associated with present treatments available for neuropathic pain and holds the promise of returning many patients to a higher quality of life with reduced need for pain medication.

Accordingly, it is the object of the present invention to provide a treatment for neuropathic pain that reduced the amount of medication required for treatment. It was a further object of the present invention to provide a treatment for neuropathic pain that reduced or avoided complications due to side effects, preferably reduced side effects due to CNS penetration. It was a further object of the present invention to provide a treatment for neuropathic pain with a reduced chance of dependence or addiction.

In addition, current therapeutic approaches provide only symptomatic relief through continuous dosing, failing to address underlying immunopathology. Another aspect of the present invention relates Immune Resolution Therapy (IRT) with AP-325, an intermittent treatment paradigm that exploits the dual nature of immune cells in neuropathic pain to shift immune phenotypes from pathological to resolving states.

This therapeutic strategy uses AP-325's dual mechanism—DHODH inhibition for selective immunomodulation and GABA modulation for immediate analgesia—through cyclic dosing that enables both suppression of inflammatory cells and subsequent phenotypic rebalancing toward regulatory immunity. The mechanistic foundation for intermittent dosing rests on three pillars: (1) the established dual nature of immune cells as both drivers and resolvers of chronic pain, (2) the requirement for drug-free intervals to enable immune resolution and phenotypic shift, and (3) precedent from cladribine's immune reconstitution therapy in MS.

### The Immune Foundation of Chronic Neuropathic Pain

The understanding of neuropathic pain has evolved from being conceptualized as a purely neuronal disorder to recognition as a complex neuroimmune condition. Austin and Moalem-Taylor (2010) fundamentally reframed neuropathic pain as a disorder involving critical interactions between neurons, inflammatory immune cells, immune-like glial cells, and cytokines. Said review established that peripheral nerve injury provokes robust immune responses at multiple anatomical sites—the injured nerve, dorsal root ganglia (DRG), and spinal cord—with the balance between pro-inflammatory and anti-inflammatory mechanisms determining whether pain chronicity develops.

The immune contribution to neuropathic pain is multifaceted and temporally dynamic. Following peripheral nerve injury, complement system activation initiates early immune responses, with C3 deposition occurring as early as 6 hours post-injury and complement components C3a and C5a serving as leukocyte chemoattractants (Austin & Moalem-Taylor, 2010). This is followed by sequential recruitment of mast cells, neutrophils, and macrophages to sites of nerve damage. Critically, T and B lymphocytes infiltrate injured nerves and DRG, where they contribute to chronic pain states through multiple mechanisms including pro-inflammatory cytokine secretion, autoantibody production, and direct sensitization of nociceptors.

### The Adaptive Immune System in Pain Chronification

Recent evidence has firmly established that adaptive immunity, particularly T cells and B cells, plays essential roles in both the development and maintenance of chronic neuropathic pain. Fiore et al. (2023) demonstrated that regulatory T cells (Tregs) are critical for pain resolution, with Treg depletion increasing mechanical pain hypersensitivity and delaying recovery after peripheral nerve injury in both males and females. Conversely, increasing Tregs through adoptive transfer reduces pain hypersensitivity in multiple models including peripheral nerve injury, experimental autoimmune neuritis, and chemotherapy-induced neuropathy.

The B cell contribution to neuropathic pain involves both antibody-dependent and antibody-independent mechanisms. Fiore et al. (2025) revealed that B cells contribute to mechanical allodynia through secretion of autoreactive IgG that binds to neuronal antigens, forming immune complexes that engage Fc gamma receptors (FcγR) in DRG and spinal cord. Importantly, this study demonstrated that all traumatic peripheral nerve injuries (chronic constriction injury, nerve crush, spared nerve injury, spinal nerve ligation) drive secretion of autoreactive IgG from B cells, though the functional consequences depend on whether cognate FcγRs are upregulated in pain pathways—a finding that underscores the complexity of immune-mediated pain mechanisms.

### The Dual Nature of Immune Cells in Pain

A transformative insight in pain neurobiology is that immune cells exhibit phenotypic plasticity, capable of adopting either pathological or resolving roles depending on their microenvironment and activation state. Fiore et al. (2023) elegantly demonstrated this duality through studies of Tregs, showing that these cells can modulate both peripheral immunity and spinal glial populations to alleviate neuropathic pain. After intrathecal delivery, activated Tregs migrate to the meninges and drain into peripheral lymphatic organs, where they expand endogenous Treg populations and shift the balance from inflammatory Th1/Th17 cells toward regulatory phenotypes.

Similarly, macrophages display remarkable plasticity, transitioning between pro-inflammatory (M1-like) and anti-inflammatory (M2-like) phenotypes. B cells likewise exhibit functional heterogeneity, with inflammatory B cell subsets secreting pro-inflammatory antibodies and cytokines, while regulatory B cells (Bregs) produce IL-10 and TGF-β to suppress excessive immune responses. This phenotypic plasticity represents a critical therapeutic opportunity: rather than eliminating immune cells, interventions can shift their functional states from pathological to resolving.
The mechanistic basis for this plasticity involves multiple factors including:
Cytokine milieu (pro-inflammatory IL-17, IFN-γ, TNF-α versus anti-inflammatory IL-10, TGF-β)
Metabolic state and availability of biosynthetic precursors
Antigen presentation and co-stimulatory signals
Temporal dynamics of immune activation and resolution

### DHODH Inhibition and Selective Lymphocyte Modulation

Without the intention of being bound by theory, AP-325 functions as an inhibitor of dihydroorotate dehydrogenase (DHODH), a key enzyme in de novo pyrimidine synthesis. DHODH catalyzes the rate-limiting step in pyrimidine biosynthesis, converting dihydroorotate to orotate within the mitochondrial inner membrane. This pathway is essential for rapidly proliferating cells, as de novo synthesis provides the nucleotides required for DNA replication and RNA transcription.

The selectivity of DHODH inhibition for activated lymphocytes arises from fundamental differences in metabolic requirements between resting and activated immune cells. Resting lymphocytes maintain minimal proliferation and can rely on the salvage pathway for nucleotide requirements, utilizing preformed nucleosides and nucleobases from cell turnover. In contrast, activated T and B cells undergo rapid clonal expansion following antigen recognition, increasing their proliferation rate 10-100 fold. This dramatic proliferative burst creates nucleotide demand that exceeds salvage pathway capacity, rendering activated lymphocytes dependent on de novo pyrimidine synthesis (Williamson et al., 1995).

Without the intention of being bound by theory, it is held that by inhibiting DHODH, AP-325 selectively constrains the proliferation of activated T and B cells without affecting resting or memory lymphocytes. This cytostatic (rather than cytotoxic) mechanism represents a critical distinction from cytotoxic immunosuppressants. Activated pathological immune cells—including Th1, Th17, and inflammatory B cells—that are actively proliferating in response to neuronal antigens and tissue damage will be preferentially suppressed. Resting immune cells and memory cells, which rely on salvage pathways, remain functionally intact, preserving protective immunity.

### GABAA Receptor Modulation and Immediate Analgesia

In addition to DHODH inhibition and without the intention of being bound by theory, AP-325 functions as a positive allosteric modulator of GABAA receptors, providing a second mechanism for pain control. GABAA receptors are ligand-gated chloride channels widely distributed throughout the central nervous system, including substantial expression in spinal dorsal horn neurons and DRG neurons. Activation of GABAA receptors induces chloride influx, hyperpolarizing neurons and reducing their excitability.

In the context of neuropathic pain and without the intention of being bound by theory, enhanced GABAergic inhibition provides multiple therapeutic benefits:
Direct inhibition of hyperexcitable nociceptors in DRG and spinal dorsal horn
Reduction of central sensitization by dampening spinal nociceptive transmission
Immediate analgesic effect during the treatment phase (T1)
Symptomatic relief while immune modulation mechanisms develop

The GABAA modulatory effect of AP-325 is mechanistically distinct from the DHODH-mediated immunomodulation, operating on different timescales and through independent pathways. While DHODH inhibition requires days to suppress lymphocyte proliferation and weeks to enable phenotypic shifts, GABAA modulation provides rapid (hours) symptomatic benefit. This dual-mechanism approach addresses both the immediate need for pain relief and the long-term goal of immune system rebalancing.

### Cytostatic Advantage

Without the intention of being bound by theory, the effect of AP-325's DHODH inhibition is cytostatic rather than cytotoxic. This distinction has profound implications for intermittent dosing strategies. Cytotoxic agents, such as cladribine, induce profound lymphocyte depletion through direct cell death, creating an immune “reset” that requires months to years for full reconstitution. While effective, this approach carries significant infection risk and requires extended drug-free intervals for immune recovery. AP-325's cytostatic mechanism transiently arrests lymphocyte proliferation without inducing cell death. During DHODH inhibition, activated lymphocytes enter cell cycle arrest, remaining viable but unable to expand clonally. Upon drug clearance, this antiproliferative pressure is released, enabling immune system recovery on a shorter timescale (weeks rather than months). The preserved viability of lymphocytes during treatment, combined with rapid clearance kinetics, enables more flexible dosing strategies with shorter drug-free intervals than would be possible with cytotoxic approaches.

Problems Associated with Continual Dosing

Conventional immunosuppressive therapy for immune-mediated disorders typically employs continuous daily dosing to maintain therapeutic drug levels and sustained immune suppression. While this approach effectively controls symptoms in conditions like rheumatoid arthritis and inflammatory bowel disease, it suffers from fundamental limitations in the context of neuropathic pain:

Continuous DHODH inhibition maintains constant antiproliferative pressure on all dividing lymphocytes, preventing both pathological and regulatory cells from expanding. This steady-state suppression prohibits the phenotypic shift from inflammatory to regulatory immunity that is essential for durable pain resolution.

These problems fundamentally arise from treating neuropathic pain as a static condition requiring perpetual suppression, rather than recognizing it as a dynamic immune dysregulation amenable to rebalancing. Continuous dosing maintains the status quo of immune suppression but cannot shift the immune system toward a self-sustaining state of resolution.

### Intermittent Dosing: The Therapeutic Cycle(s)

Immune Resolution Therapy with AP-325 employs intermittent dosing cycle(s) comprising a treatment phase (T1) and a drug-free recovery phase (T2). This cyclical approach involves both phases working sequentially together as therapeutically essential components.

### Treatment Phase (T1): Disruption of Pathological Feedback

During T1 (10-84 days depending on disease severity), DHODH inhibition arrests proliferation of activated inflammatory lymphocytes while GABAA modulation provides immediate analgesia. The pathological feedback loop—neuronal damage→antigen release→immune activation→cytokine secretion→neuronal sensitization→chronic pain—is interrupted at the immune activation step.

Without the intention of being bound by theory, T1 achieves:
Reduction of Th1/Th17 cells: Pro-inflammatory CD4+ T cell subsets that secrete IFN-γ and IL-17, directly sensitizing nociceptors and recruiting inflammatory cells, undergo cell cycle arrest.
Suppression of inflammatory B cells: B cell clones producing autoreactive IgG against neuronal antigens (as demonstrated by Fiore et al., 2025) cannot expand, reducing autoantibody titers.
Decreased pro-inflammatory cytokines: With fewer activated inflammatory cells, levels of TNF-α, IL-1β, IL-6, and IL-17 decline.
Reduced nociceptor sensitization: Lower cytokine burden and direct GABAergic inhibition decrease neuronal hyperexcitability.
Pain relief: The combination of reduced inflammation and enhanced inhibitory neurotransmission provides measurable analgesic benefit.
   Importantly, T1 does not eliminate lymphocytes—it temporarily constrains their proliferation, preserving the cellular substrate for subsequent phenotypic rebalancing during T2.

### Drug-Free Phase (T2): Active Immune Resolution

Without the intention of being bound by theory, T2 (7-200 days, calibrated to T1 duration and disease severity) represents the mechanistically essential recovery and rebalancing phase. This is not passive “washout” but an active therapeutic period during which multiple resolution mechanisms engage:

### Drug Clearance and Proliferative Release (Days 1-7 of T2):

AP-325 clears from circulation with an elimination half-life allowing complete clearance within the first week of T2. As tissue drug levels decline below the threshold for DHODH inhibition, the antiproliferative block is released, permitting lymphocyte proliferation to resume.

### Homeostatic Immune Recovery (Days 7-35 of T2):

Following the period of constrained proliferation, the immune system undergoes homeostatic expansion/proliferation to restore lymphocyte populations and diversity. Importantly, this recovery process is not a simple return to the pre-treatment inflammatory state.

### Multiple Mechanisms Bias Recovery Toward Regulatory Phenotypes

Homeostatic Proliferation after DHODH inhibition: Following a period of reduced proliferation, regulatory T cells (Tregs) preferentially expand compared to effector T cells, a phenomenon well-documented in multiple contexts (Fiore et al., 2023; Wu et al., 2023).
Altered Cytokine Milieu: The reduced inflammatory burden during T1 shifts the tissue cytokine environment. IL-2, which drives Treg expansion, becomes relatively more available as inflammatory effector cells are suppressed. The decreased competition for IL-2 favors Treg proliferation during T2 recovery.
Thymic-Independent Peripheral Expansion: In adults, most immune reconstitution occurs through peripheral expansion of existing cells rather than thymic output. During recovery from transient suppression, memory and regulatory populations that were less affected by DHODH inhibition (due to lower baseline proliferation rates) serve as the founding population for expansion, skewing the recovering immune system toward regulatory phenotypes.

### Phenotypic Maturation and Stabilization (Days 35-84+ of T2):

As lymphocyte numbers recover, the newly expanded populations undergo functional maturation. Critical phenotypic shifts occur:
Treg Consolidation: Expanded Treg populations stabilize their suppressive phenotype, expressing high levels of FoxP3, CTLA-4, and secreting IL-10 and TGF-β.
Regulatory B Cell (Breg) Expansion: B cells recovering from suppression preferentially adopt regulatory phenotypes, contributing to IL-10 production.
Resolution-Phase Mediators: The shifting immune milieu favors production of specialized pro-resolving mediators (resolvins, lipoxins, protectins).
M2 Macrophage Polarization: As Tregs expand and secrete IL-10 and TGF-β, tissue macrophages undergo phenotypic polarization from M1 (pro-inflammatory) to M2 (tissue-remodelling, anti-inflammatory) phenotypes, as demonstrated by Fiore et al. (2023)
   Sustained Benefit without Drug:

Without the intention of being bound by theory, it is held that effective IRT provides pain relief obtained during T1 that is maintained or even improved during 12, despite absence of drug. This persistence of benefit without ongoing pharmacological suppression represents direct evidence of immune system rebalancing rather than mere symptomatic control. The resolution phase generates a self-sustaining shift toward regulatory immunity that continues to suppress pathological immune responses even after drug clearance.

### Cumulative Effects Across Multiple Cycles

Without the intention of being bound by theory, it is held that the treatment cycle (T1→T2) shifts the immune system incrementally toward a regulatory-dominant state. The therapeutic benefit is cumulative:
Initial treatment cycle results in reduction of inflammatory cell burden with first phenotypic shift toward regulatory immunity. Pain improves during T1 and is maintained during T2. If not sufficiently realized in the first cycle, a further treatment cycle depletes pathological clones that expanded during the chronic pain development phase. The regulatory cell population, now larger relative to inflammatory cells, exerts stronger suppressive effects. Pain continues to improve. When not sufficiently realized in the first or second treatment cycles such as in severe cases, one or more further treatment cycle(s) follows the second cycle and progressive consolidation of regulatory dominance establishes a new immune homeostasis. The ratio of regulatory to effector cells reaches a threshold where inflammatory responses to neuronal antigens are effectively suppressed by endogenous regulatory mechanisms. At this stage, pain resolution may be self-sustaining without further treatment.

### SUMMARY OF THE INVENTION

Said objects are solved by the aspects of the invention as defined in the claims, described in the description, and illustrated in the Examples and Figures.

In a first aspect, the invention provides a method of treating neuropathic pain in human patients comprising
a treatment cycle comprising:
   administering a compound according to formula (I), or an isomer thereof, or a tautomer thereof, or an isotope thereof, over a period T¹; formula (I) having the structure of:

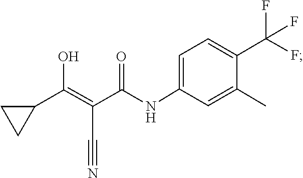 [SMILES: Cc1cc(NC(=O)/C(C#N)=C(\O)C2CC2)ccc1C(F)(F)F]

discontinuing the administration of the compound according to formula (I) for a period T² that immediately follows T¹;
obtaining pain reduction over the total sum of the periods T¹ and T² of the treatment cycle.

In another aspect, the invention provides a method of immune resolution therapy (IRT) comprising
a treatment cycle comprising:
   administering a compound according to formula (I), or an isomer thereof, or a tautomer thereof, or an isotope thereof, over a period T¹; formula (I) having the structure of:

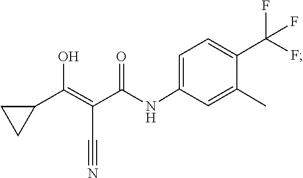 [SMILES: Cc1cc(NC(=O)/C(C#N)=C(\O)C2CC2)ccc1C(F)(F)F]

inhibiting of dihydroorotate dehydrogenase (DHODH) during T¹;
modulating GABA receptors during T¹;
discontinuing the administration of the compound according to formula (I) for a period T² that immediately follows T¹;
clearing the compound according to formula (I) during 12;
modulating lymphocyte subpopulations during T² relative to the last day of T¹, optionally determined by flow cytometry or Fluorescence-Activated Cell Sorting (FACS);
obtaining pain reduction over the total sum of the periods T¹ and T² of the treatment cycle.

In a further aspect, the invention provides a method of treating neuropathic pain in human patients comprising
a treatment cycle comprising:
   administering a compound according to formula (I) over a period T¹; formula (I) having the structure of:

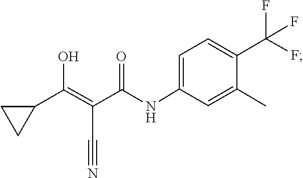 [SMILES: Cc1cc(NC(=O)/C(C#N)=C(\O)C2CC2)ccc1C(F)(F)F]

discontinuing the administration of the compound according to formula (I) for a period T² that immediately follows T¹;
wherein period T²≥period T¹ or period T²≥2×period T¹;
   obtaining pain reduction over the total sum of the periods T¹ and T² of the treatment cycle;
wherein for the duration of T², the obtained pain reduction is about equal to or greater than the maximum pain reduction measured during T¹.

In a further aspect, the invention provides a method of treating neuropathic pain in human patients comprising
a treatment cycle comprising:
   administering a compound according to formula (I) over a period T¹; formula (I) having the structure of:

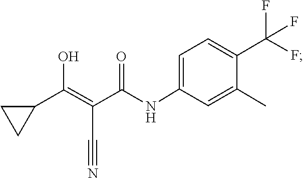 [SMILES: Cc1cc(NC(=O)/C(C#N)=C(\O)C2CC2)ccc1C(F)(F)F]

discontinuing the administration of the compound according to formula (I) for a period T² that immediately follows T¹;
wherein period T²≥period T¹ or period T²≥2×period T¹;
   obtaining pain reduction over the total sum of the periods T¹ and T² of the treatment cycle;
wherein for the duration of T², the obtained pain reduction is about equal to or greater than the maximum pain reduction measured during T¹;
wherein at least 20%, at least 30% or at least 35% of patients undergoing the treatment cycle have a successful treatment determined at the end of the period of T² according to the Patient Global Impression Of Change (PGIC) scale.

The Patient Global Impression of Change (PGIC) relates the response that best describes the change in the overall status since the patient started treatment, the score choices are:
(1) Very much improved
(2) Much improved
(3) Minimally improved
(4) No change
(5) Much worse
(6) Much worse
(7) Very much worse.
   As defined herein, a successful treatment is a mean PGIC score of “very much improved” or “Much improved”.

In a further aspect, the invention provides a method of treating neuropathic pain in human patients comprising
a treatment cycle comprising:
   administering a compound according to formula (I) over a period T¹; formula (I) having the structure of:

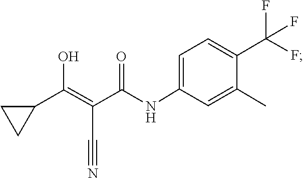 [SMILES: Cc1cc(NC(=O)/C(C#N)=C(\O)C2CC2)ccc1C(F)(F)F]

discontinuing the administration of the compound according to formula (I) for a period T² that immediately follows T¹;
wherein period T²≥period T¹ or period T²≥2×period T¹;
   obtaining pain reduction over the total sum of the periods T¹ and T² of the treatment cycle;
wherein for the duration of T², the obtained pain reduction is about equal to or greater than the maximum pain reduction measured during T¹;
wherein an improvement in the mean value of the Hospital Anxiety and Depression Score (HADS), determined relative to a baseline level of the mean value according to the Hospital Anxiety and Depression Score (HADS) measured immediately before the period T¹ is begun, is obtained concomitant with pain reduction over the duration of period T² of the treatment cycle.

In a further aspect, the invention provides a method of treating neuropathic pain in human patients comprising
a treatment cycle comprising:
   administering a compound according to formula (I) over a period T¹; formula (I) having the structure of:

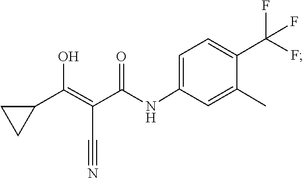 [SMILES: Cc1cc(NC(=O)/C(C#N)=C(\O)C2CC2)ccc1C(F)(F)F]

discontinuing the administration of the compound according to formula (I) for a period T² that immediately follows T¹;
wherein period T²≥period T¹ or period T²≥2×period T¹;
   obtaining pain reduction over the total sum of the periods T¹ and T² of the treatment cycle;
wherein for the duration of T², the obtained pain reduction is about equal to or greater than the maximum pain reduction measured during T¹;
wherein an absolute improvement in the mean value according to the Daily Sleep Interference Scale (DSIS), determined relative to a baseline level of the mean value according to the Daily Sleep Interference Scale (DSIS) measured immediately before the period T¹ is begun, is obtained concomitant with pain reduction over the duration of period T² of the treatment cycle;
wherein an improvement in the mean value of the Hospital Anxiety and Depression Score (HADS), determined relative to a baseline level of the mean value according to the Hospital Anxiety and Depression Score (HADS) measured immediately before the period T¹ is begun, is obtained concomitant with pain reduction over the duration of period T² of the treatment cycle;
wherein patients undergoing the treatment cycle have a successful treatment determined at the end of the period of T² according to the Patient Global Impression Of Change (PGIC) scale, wherein a treatment is successful if a patient reports a much improved to very much improved overall status according to the Patient Global Impression Of Change (PGIC) scale;
wherein less than 30% of patients undergoing the treatment cycle have had a Treatment Emergent Adverse Event (TEAE) determined at the end of the period of 12.

In a further aspect, the invention provides a method of treating neuropathic pain in human patients comprising
a treatment cycle comprising:
   administering a compound according to formula (I) over a period T¹; formula (I) having the structure of:

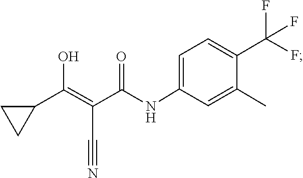 [SMILES: Cc1cc(NC(=O)/C(C#N)=C(\O)C2CC2)ccc1C(F)(F)F]

discontinuing the administration of the compound according to formula (I) for a period T² that immediately follows T¹;
wherein period T²≥period T¹;
   obtaining pain reduction over the total sum of the periods T¹ and T² of the treatment cycle;
wherein for the duration of T², the obtained pain reduction is about equal to or greater than the maximum pain reduction measured during T¹;
wherein none of the patients undergoing treatment experience drug withdrawal, drug withdrawal syndrome, or substance withdrawal syndrome

In a further aspect, the invention provides a compound according to formula (I), or an isomer thereof, or a tautomer thereof, or an isotope thereof, having the structure of:

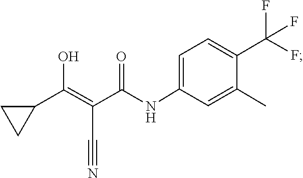 [SMILES: Cc1cc(NC(=O)/C(C#N)=C(\O)C2CC2)ccc1C(F)(F)F]

for use in treating neuropathic pain in a method according to any one of items 1 to 116.

In a further aspect, the invention relates the use of a compound according to formula (I), or an isomer thereof, or a tautomer thereof, or an isotope thereof; having the structure of:

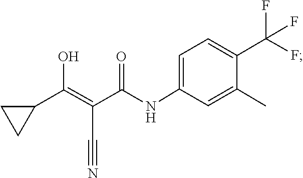 [SMILES: Cc1cc(NC(=O)/C(C#N)=C(\O)C2CC2)ccc1C(F)(F)F]

for preparing a pharmaceutical composition for treating a human patient suffering from neuropathic pain, wherein the use comprises a method according to any one of items 1 to 116.

### BRIEF DESCRIPTION OF THE DRAWINGS

The invention will be better understood with reference to the detailed description when considered in conjunction with the non-limiting examples and the drawings, in which:

FIG. 1 shows the mean percentage change in the 5-day average PI-NRS scores for Day 5, 10, 15, 20, 25, 30 and 35 are displayed with the standard error of the mean as error bars.

FIG. 2A shows the responder rate (at 30%, 50% and 70% reduction, binary variable on patient level) compared between treatments on Day 10.

FIG. 2B shows the day 35 results and calculated from PI-NRS scores.

FIG. 3 shows the time to first rescue medication presented by treatment group. The number of patients that. intake of rescue medication and patients that are censored (e.g. premature withdrawal) are displayed. The time-to-event analysis is illustrated using Kaplan-Meier plots.

FIG. 4 shows the mean change in the 5-day average of the Daily Sleep Interference Scale (DSIS) scores for Day 5, 10, 15, 20, 25, and 35 in a line graph including standard error of the mean as error bars.

FIG. 5 shows the differences in change from Baseline to Day 10 and Day 35 in the the hospital anxiety and depression scale (HADS). Total scores are compared between treatments using linear regression.

FIG. 6A shows the results of the Patient Global Impression of Change (PGIC) survey of the proportion of patients that reports a treatment success (the mean of patients reporting much better or very much better improvement) on Day 10 is comparable between AP-325 treated patients and placebo treated patients.

FIG. 6B shows that there is a statistically significant difference between AP-325 treated patients and placebo treated patients in the proportion of patients that reports a treatment success.

FIG. 7 shows the secondary data analysis by post-hoc analyses estimated group-mean PI-NRS scores over the follow-up time.

### DETAILED DESCRIPTION OF THE INVENTION

In a first aspect, the present invention relates a method of treating neuropathic pain in human patients comprising a treatment cycle comprising: administering a compound according to formula (I), or an isomer thereof, or a tautomer thereof, or an isotope thereof, over a period T¹; formula (I) having the structure of:

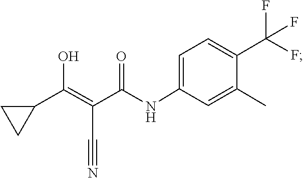 [SMILES: Cc1cc(NC(=O)/C(C#N)=C(\O)C2CC2)ccc1C(F)(F)F]

discontinuing the administration of the compound according to formula (I) for a period T² that immediately follows T¹; obtaining pain reduction over the total sum of the periods T¹ and T² of the treatment cycle.

As defined herein, a compound according to formula (I) is frequently referred to as AP-325 in the present disclosure and has the structure of:

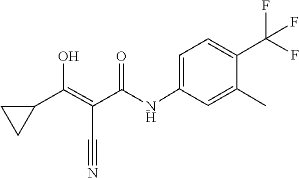 [SMILES: Cc1cc(NC(=O)/C(C#N)=C(\O)C2CC2)ccc1C(F)(F)F]

that corresponds to the name (Z)-2-cyano-3-cyclopropyl-3-hydroxy-N-(3-methyl-4-(trifluoromethyl)phenyl)acrylamide or simply 2-cyano-3-cyclopropyl-3-hydroxy-N-[3-methyl-4-(trifluoromethyl)phenyl]prop-2-enamide (IUPAC names) and has the assigned CAS number 147076-36-6 for the sodium salt of the conjugate base thereof. Regarding isotopes of AP-325, the skilled person is aware that isotopes of hydrogen such as deuterium have a natural abundance and each position occupied by a hydrogen of the compound according to formula (I) has an associated natural abundance of deuterium bound in place of the isotope of hydrogen. As such, deuteration of each hydrogen position of the compound according to formula I has an associated natural abundance fraction and multiple deuterium's can be bound to a single molecule in place of multiple hydrogens. Chemical replacement of hydrogen with deuterium is also well known within the art including the effects therefrom including improved PK of isotopically labelled positions of said compound. Tautomers of AP-325 as well as salts of the deprotonated conjugate base are also possible and will be immediately apparent to one skilled in the art of organic chemistry.

### Administering a Compound According to Formula (I)

As defined herein, administering a compound according to formula (I), or an isomer thereof, or a tautomer thereof, or an isotope thereof, over a period T¹ means to dose a patient with a compound according to formula (I) (AP-325), preferably intermittently dosed, for the duration of the period T¹. In this sense, administering a compound according to formula (I) would be given its usual meaning of dosing a patient including all routes of administration such oral (per os) or intravenous (IV) as non-limiting examples including variations thereof. It is preferred that the compound according to formula (I) is administered orally. However, the skilled person will also realize that intravenous administration may be advantageous in some cases and may apply this form of dosing a compound according to formula (I).

It is preferred that the compound according to formula (I) is administered orally. The present invention is not particularly limited to the form of oral administration of AP-325 and the compound according to formula (I) can be administered in the form of a capsule, tablet, granules, drop or liquid. It is preferred that the compound according to formula (I) is administered in the form of a capsule or tablet. It is further preferred that an individual capsule or tablet comprises the compound according to formula (I) in an amount in the range of from 2.5 to 250 mg, preferably 2.5 to 100 mg, more preferably 5 to 50 mg, more preferably 10 to 30 mg, preferably in the range of from 20 to 30 mg, more preferably being about 25 mg. However, in general embodiments it is more preferred that an individual capsule or tablet comprises the compound according to formula (I) in an amount of about 50 mg or about 100 mg, preferably about 100 mg.

It is also preferred that the compound according to formula (I) is administered as a pharmaceutical composition. In some embodiments, the pharmaceutical composition comprises one or more excipients, preferably one or more pharmaceutically acceptable excipients. As defined herein, pharmaceutically acceptable means suitable for use in contact with the tissues of humans and animals without undue toxicity, irritation, allergic response, and the like. In some embodiments, the one or more excipients are selected from the list consisting of solvent, binders, surfactants, stabilizers, fillers, lubricants, bulking agents, anti-caking agents, glidants, preservatives and buffering agents, preferably selected from the group consisting of fillers, binders, glidants, lubricants, and disintegrants. In more detailed embodiments, the excipients comprise, preferably consist of, poloxamer 407, sodium glycocholate, hypromellose (hydroxypropylmethylcelluose), isomalt and talc. In related detailed embodiments, the compound according to formula (I) is in the form of a salt, preferably a pharmaceutically acceptable salt, more preferably in the form of a sodium salt. As defined herein, a pharmaceutically acceptable salt are those that form non-toxic base addition salts, i.e., salts containing pharmacologically acceptable cations such as, but not limited to, alkali metal or alkaline earth metal salts and the calcium, magnesium, sodium or potassium salts in particular, preferably being sodium. Further suitable excipients, salts and compositions are provided in WO2015140081 A1 and US2008/0241070A1.

Concerning the dosage amount per the weight of the patient, in general it is preferred that the administering of the patient with the compound according to formula (I) over a period T¹ is with an effective amount. In more detailed embodiments, the administering of the patient with the compound according to formula (I) over a period T¹ is with an amount, preferably an effective amount, according to claim 26, in the range of from 0.05 mg of the compound per 1 kg of the patient to 5.0 mg of the compound per 1 kg of the patient, preferably 0.1 mg of the compound per 1 kg of the patient to 5.0 mg of the compound per 1 kg of the patient, more preferably in the range of from 0.25 mg of the compound per 1 kg of the patient to 3.0 mg of the compound per 1 kg of the patient, more preferably in the range of from in 0.7 mg of the compound per 1 kg of the patient to 2.7 mg of the compound per 1 kg of the patient, more preferably in the range of from in 0.9 mg of the compound per 1 kg of the patient to 2.5 mg of the compound per 1 kg of the patient, more preferably in the range of from in 1.0 mg of the compound per 1 kg of the patient to 2.0 mg of the compound per 1 kg of the patient. The administering of the patient with the compound according to formula (I) over a period T¹ is with an amount range of from 0.05 mg of the compound per 1 kg of the patient to 5.0 mg of the compound per 1 kg of the patient includes 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95 and 5 mg of the compound per 1 kg of the patient.

Regarding the frequency of administering, the present invention is not particularly limited. In general, it is preferred that the patient is dosed daily with a compound according to formula (I) over the period T¹. In some embodiments, the compound according to formula (I) is administered one or more times per day, preferably two to three times per day. In more preferred embodiments, the compound according to formula (I) is administered once per day.

As used herein, at the end of T¹ or T² shall be interpreted as being on the final day of said period, preferably after the final dose is administered and peak AUG of the compound according to formula I has been achieved in the patient as determined by HPLC-MS analysis or LC-MS/MS, preferably LC-MS/MS of serum. Likewise, at the beginning of T¹ or T² shall be interpreted as being on the first day of said period, preferably after the first dose is administered in T¹ for period T¹ or the day following the final day of T¹ for period T².

Considering the duration of administering a compound according to formula (I), said duration is defined herein as the period T¹ which is given in days. In general, the period T¹ is shorter in duration than the period T² during which administration of compound according to formula (I) is discontinued. In general embodiments, period T²≥period T¹. In more specific embodiments, period T²≥2×period T¹, in other words period T² is greater than or equal to twice the value of the period T¹. In some embodiments, period T¹≤20 days, preferably period T¹≤17 days, more preferably period T¹≤15 days. A period T¹≤20 days includes 1, 2, 3, 4, 5, 6, 7, 8, 9, 10, 11, 12, 13, 14, 15, 16, 17, 18, 19 and 20 day(s). In related embodiments period T¹ is in the range of from 1 to 100 days, preferably 1 to 84 days, more preferably 1 to 42 days, more preferably 1 to 28 days, more preferably 1 to 21 days, more preferably in the range of from 2 to 19 days, more preferably in the range of from 3 to 18 days, more preferably in the range of from 4 to 17 days, more preferably in the range of from 5 to 16 days, more preferably in the range of from 6 to 15 days, more preferably in the range of from 7 to 14 days, more preferably in the range of from 8 to 13 days, more preferably in the range of from 9 to 12 days, more preferably in the range of from 10 to 11 days. Period T¹ is in the range of from 1 to 100 days includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100 day(s).

### Discontinuing the Administration of the Compound According to Formula (I) for a Period T²

In general, discontinuing the administration of the compound according to formula (I) for a period T² that immediately follows T¹ means that no further compound according to formula (I) is dosed for the duration of the period T² and that the period T² immediately begins when period T¹ ends. As noted above and in general, the period T² is longer in duration than period T¹ and the effect of pain reduction is also equal to or higher than the pain reduction obtained during period T¹. It is preferred that period T²≥1 day, preferably period T²≥2 days, more preferably period T²≥3 days, more preferably period T²≥4 days, more preferably period T²≥5 days, more preferably period T²≥10 days, more preferably period T²≥15 days, more preferably period T²≥17 days, more preferably period T²≥20 days, more preferably period T²≥21 days, more preferably period T²≥28 days, more preferably period T²≥30 days, more preferably period T²≥34 days, more preferably period T²≥40 days, more preferably period T²≥42 days, more preferably period T²≥56 days more preferably period T²≥84 days. In related embodiments, period T² is in the range of from 1 to 200 days, preferably in the range of from 1 to 168 days, more preferably in the range of from 1 to 100 days, preferably in the range of from 1 to 84 days, preferably in the range of from 1 to 56 days, preferably in the range of from 1 to 50 days, preferably in the range of from 1 to 42 days, more preferably in the range of from 1 to 40 days, more preferably in the range of from 4 to 38 days, more preferably in the range of from 6 to 36 days, more preferably in the range of from 8 to 34 days, more preferably in the range of from 10 to 32 days, more preferably in the range of from 12 to 30 days, more preferably in the range of from 14 to 28 days, more preferably in the range of from 16 to 26 days, more preferably in the range of from 18 to 24 days, more preferably in the range of from 20 to 22 days. Period T² being in the range of from 1 to 200 days includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 and 200 day(s).

Concerning the amount of AP-325 present in the patients during the period T², some amount of compound is expected early during the period but this amount decreases in the patients during said period while the same if not better effects in pain reduction is observed. Tellingly, the mean amount of AP-325 on day 35 of the clinical trial was less than 0.1% of the maximum mean amount measured on the final day of T¹. It is therefore preferred that only negligible amounts (negligible amounts defined herein as 10% or less of the maximum amount measured at the end of T¹, negligible amounts preferably being 10% to >1%), preferably trace amounts (trace amounts defined herein as % or less, more preferably 0.1% or less, more preferably 0.05% or less of the maximum amount measured at the end of T¹), more preferably no detectable amount, of the compound according to formula (I) is detectable within the patients plasma on the final day of T², preferably determined by HPLC/mass spectroscopy. Likewise, it is preferred that only negligible amounts, preferably trace amounts, more preferably no detectable amount, of metabolites of the compound according to formula (I) are detectable within the patients plasma on the final day of T², and negligible amounts and trace amounts are as defined for the metabolite(s) as above for the compound according to formula (I). It is preferred that no detectable amount of the compound according to formula (I) is detectable within the patients plasma on the final day of T².

### Additional Treatment Cycles

The present invention includes further treatment cycles that may be required for neuropathic pain that returns after a first treatment cycle. In general, the method may comprise one or more additional treatment cycles. Also in general, the patients are of 18 years of age or older. Older patients in the geriatric population have, in general, lower capacity for healing damaged nerves and may require additional treatment cycles.

The additional treatment cycle is initiated in response to clinically meaningful changes in pain intensity scores of the Pain Intensity Numeric Rating Scale (PI-NRS). A change of 1 to 2 absolute points (mean value) on the Pain Intensity Numeric Rating Scale is generally accepted as being clinically meaningful, corresponding to a 20% to 35% change in the mean value of the PI-NRS. In general embodiments, the method comprises an evaluation period T^{Ev} that immediately follows period T², immediately following meaning that period T^{Ev} begins immediately when period T² ends. In general embodiments, the method comprises an evaluation period T^{Ev} that immediately follows period T². In further related embodiments, the evaluation period T^{Ev} is equal to the number of days required for the loss in absolute pain reduction, preferably the mean absolute pain reduction, to be greater than 1.0 point, preferably greater than 1.3 points, preferably greater than 1.5 points, determined relative to the obtained absolute pain reduction, preferably the obtained mean absolute pain reduction, measured on the final day of period T² and determined according to pain intensity scores of the Pain Intensity Numeric Rating Scale (PI-NRS). In related embodiments, a further treatment cycle is initiated immediately following T^{Ev}, preferably the further treatment cycle is initiated when the loss in absolute pain reduction, preferably the mean absolute pain reduction, is greater than 1.0 point, preferably greater than 1.3 points, preferably greater than 1.5 points determined relative to the obtained absolute pain reduction, preferably the obtained mean absolute pain reduction, measured on the final day of period 12 and determined according to pain intensity scores of the Pain Intensity Numeric Rating Scale (PI-NRS). A loss in pain reduction being synonymous with an increase in pain and it will be apparent to the skilled person that gains in pain reduction can be lost overtime and the decision to initiate a further treatment cycle when a clinically significant increase in the pain intensity scores of the Pain Intensity Numeric Rating Scale (PI-NRS) is observed. It is therefore preferred that the evaluation period TES is equal to the number of days required for the loss in pain reduction, preferably the loss in mean pain reduction, is greater than 15%, preferably greater than 20% determined relative to the obtained pain reduction, preferably the obtained mean pain reduction, measured on the final day of period T² and determined according to pain intensity scores of the Pain Intensity Numeric Rating Scale (PI-NRS). It is further preferred that a further treatment cycle is initiated immediately following T^{Ev} preferably the further treatment cycle is initiated when the loss in pain reduction is greater than 15%, preferably greater than 20% determined relative to the obtained pain reduction, preferably the obtained mean pain reduction, measured on the final day of period T² and determined according to pain intensity scores of the Pain Intensity Numeric Rating Scale (PI-NRS).

In general, the treatment cycle can be repeated, preferably as necessary for management of recurring neuropathic pain or chronic neuropathic pain

### Neuropathic Pain and Causes Thereof

Neuropathic pain has characteristics that differentiate it from other types of pain, in particular nociceptive pain (Attal, 2023). These characteristics encompass several symptoms or sensory descriptors (e.g. burning, cold pain, electric shocks, tingling, pins and needles, numbness, and itch) and are common to diverse medical conditions (e.g. post-stroke pain and diabetic painful neuropathies). In general, it is preferred that the neuropathic pain is peripheral and/or predominantly peripheral neuropathic pain or central and/or predominantly central neuropathic pain. In general, it is preferred that the neuropathic pain is peripheral neuropathy caused by damage to peripheral nerves resulting in symptoms chosen from the group consisting of numbness, tingling, autonomic dysfunction, pain, and weakness in the extremities.

Neuropathic pain in peripheral neuropathy has also been linked to axonal degeneration (Cashman, 2015) and in some embodiments, the neuropathic pain is peripheral neuropathy caused by axonal degeneration. In some embodiments, the peripheral neuropathy caused by axonal degeneration is characterized by deterioration or death of the long projections of neurons (axons). In more specific embodiments, the axonal degeneration is resultant from metabolic dysfunction, oxidative stress, mitochondrial impairment and/or disruption of axonal transport.

Toxins and xenobiotic compounds such as drugs are known to cause neuropathic pain (Jones 2020; Peters 2022). Inflammation has also been shown to play a role in some neuropathic pain studies (Bennet 2013). Small fiber neuropathy has also been shown in some neuropathic pain studies (Hovaguimian 2011). In some embodiments the neuropathic pain is peripheral neuropathy chosen from the group of causations consisting of toxins (optionally Drug-Induced Peripheral Neuropathy (DIPN) preferably resulting from alcohol and/or drug abuse), inflammation/immune-mediated, hereditary, vasculitic disease, amyloid neuropathy, sarcoidosis, connective tissue disease, thyroid dysfunction, vitamin B12 deficiency and small fiber neuropathy.

In general embodiments, the neuropathic pain is resultant from or is characterized by a cause that is selected from the group of systemic diseases, e.g. diabetic neuropathy; drug-induced lesions, e.g. neuropathy due to chemotherapy; traumatic syndrome and entrapment syndrome; lesions in nerve roots and posterior ganglia; neuropathies after HIV infections; neuralgia after Herpes infections; nerve root avulsions; cranial nerve lesions; cranial neuralgias, e.g., trigeminal neuralgia; neuropathic cancer pain; phantom pain; compression of peripheral nerves, neuroplexus and nerve roots; paraneoplastic peripheral neuropathy and ganglionopathy; complications of cancer therapies, e.g. chemotherapy, irradiation, and surgical interventions; complex regional pain syndrome; type I lesions (previously known as sympathetic reflex dystrophy]; and type II lesions (corresponding approximately to causalgia]; cerebral lesions that are predominantly thalamic; infarction, e.g. thalamic infarction or brain stem infarction; cerebral tumors or abscesses compressing the thalamus or brain stem; multiple sclerosis; brain operations, e.g. thalamotomy in cases of motoric disorders; spinal cord lesions; spinal cord injuries; spinal cord operations, e.g. anterolateral cordotomy; ischemic lesions; anterior spinal artery syndrome; Wallenberg's syndrome; and syringomyelia.

In general embodiments, the neuropathic pain is postherpetic neuralgia (caused by Herpes Zoster], root avulsions, painful traumatic mononeuropathy, painful polyneuropathy (particularly due to diabetes], central pain syndromes (potentially caused by virtually any lesion at any level of the nervous system], postsurgical pain syndromes (eg, postmastectomy syndrome, post thoracotomy syndrome, phantom pain] and/or complex regional pain syndrome (reflex sympathetic dystrophy and causalgia].

In some embodiments, the neuropathic pain is a central pain syndrome caused by spinal cord injury and/or spinal cord contusion.

In some embodiments, the neuropathic pain is idiopathic.

In general embodiments, the neuropathic pain is a chronic neuropathic pain, preferably wherein the chronic neuropathic pain is early chronic neuropathic pain characterized by persistent pain existing for a duration in the range of 4 to 12 months or the chronic neuropathic pain is persistent pain existing for a duration of 1 to 15 years.

In general embodiments, the neuropathic pain is post-surgical neuropathic pain, preferably peripheral post-surgical neuropathic pain. In some embodiments, the neuropathic pain is post-surgical neuropathic pain, preferably peripheral post-surgical neuropathic pain selected from the group consisting of breast surgery, chest surgery, hernia repair, abdominal surgery, varicose vein surgery or gynecologic surgery. In specific embodiments, the neuropathic pain is post-surgical neuropathic pain after breast surgery. In specific embodiments, wherein the neuropathic pain is post-surgical neuropathic pain after chest surgery. In specific embodiments, the neuropathic pain is post-surgical neuropathic pain after hernia repair. In specific embodiments, the neuropathic pain is post-surgical neuropathic pain after abdominal surgery. In specific embodiments, the neuropathic pain is post-surgical neuropathic pain after varicose vein surgery. In specific embodiments, the neuropathic pain is post-surgical neuropathic pain after gynecologic surgery.

### Obtaining Pain Reduction

In general, pain reduction is commonly measured by well established scoring methods that involves patients tracking pain by rating the intensity of the pain on a scale. Pain intensity scores measured according to the Pain Intensity Numeric Rating Scale (PI-NRS) is commonly used in clinical settings for quantifying reduction and gain of pain (Farrar 2001). The PI-NRS has been further broadly evaluated with the Patient Global Impression Of Change (PGIC) scale which has been shown to be clinically valid in measuring the success of pain treatment (Farrar 2001). Other scales have also been evaluated alongside the PI-NRS and also shown to be clinically valid such as the Hospital Anxiety and Depression Score (HADS) (see Zigmond 1983). Further validated measures of pain reduction relate to the Daily Sleep Interference Scale (DSIS) (see Vernon M K, 2008).

Accordingly, a concept common to the scales and different measurements of pain use a baseline established prior to beginning treatment, involving averaging the pain scores on the respective scales in questionnaires or pain dairies. Likewise, an absolute value or point as used herein refers to a point scored on the aforementioned scales and a mean absolute value or point is the average of several points scored on the respective scale. In as defined herein, all values that are discussed in terms of the respective pain scales are mean values obtained from averaging scores on said scales unless specifically mentioned.

In general embodiments, obtaining pain reduction over the total sum of the periods T¹ and T² of the treatment cycle is determined according to pain intensity scores of the Pain Intensity Numeric Rating Scale (PI-NRS). However, initial pain reduction early during period T¹ is lower than the maximum value obtained at the end of period T¹. Surprisingly, the pain reduction during the period T² is greater than or about equal to the maximum value in pain reduction obtained during period T¹. In general embodiments, for the duration of T², the obtained pain reduction is about equal to or greater than the maximum pain reduction measured during T¹, preferably determined according to pain intensity scores of the Pain Intensity Numeric Rating Scale (PI-NRS). Thus in some embodiments, for the duration of T², the obtained pain reduction is greater than 15%, preferably greater than 20% determined relative to a baseline level of pain measured immediately before the period T¹ is begun, preferably measured according to pain intensity scores of the Pain Intensity Numeric Rating Scale (PI-NRS). In related specific embodiments, the obtained pain reduction is greater than 25%, preferably greater than 26%, more preferably greater than 30%, more preferably greater than 35% preferably, more preferably greater than 40%, more preferably greater than 45% preferably measured according to pain intensity scores of the Pain Intensity Numeric Rating Scale (PI-NRS). In further related embodiments, the obtained pain reduction is in the range of from 15% to 100%, preferably is in the range of from 20% to 100%, more preferably in the range of from 21% to 90%; more preferably in the range of from 22% to 80%; more preferably in the range of from 23% to 70%; more preferably in the range of from 24% to 60%; more preferably in the range of from 25% to 50%, more preferably in the range of from 25% to 40% more preferably in the range of from 25% to 35%. The obtained pain reduction is in the range of from 15% to 100% includes 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In general, the maximum obtained pain reduction for T¹ is greater than 15%, preferably greater than 20%, more preferably greater than 25%, determined relative to the baseline level of pain measured immediately before the period T¹ is begun, preferably measured according to pain intensity scores of the Pain Intensity Numeric Rating Scale (PI-NRS). In related embodiments, the obtained pain reduction is in the range of from 15% to 100%, preferably is in the range of from 20% to 100%, more preferably in the range of from 21% to 90%, more preferably in the range of from 22% to 80%, more preferably in the range of from 23% to 70%, more preferably in the range of from 24% to 60%, more preferably in the range of from 25% to 50%, more preferably in the range of from 25% to 40% more preferably in the range of from 25% to 35%, more preferably in the range of from 25% to 30%. The obtained pain reduction is in the range of from 15% to 100% includes 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In general, the absolute pain reduction, preferably the mean absolute pain reduction, over the total sum of the periods T¹ and T² of the treatment cycle is greater than 1.0 point, preferably greater than 1.3 points, preferably greater than 1.5 points, more preferably greater than 2 points, more preferably greater than 2.5 points, more preferably in the range of 1.0 to 5.0 points, more preferably in the range of 2.0 to 4.5 points, more preferably in the range of 2.5 to 4.0 points, more preferably in the range of 2.5 to 3.5 points, more preferably in the range of 2.5 to 3.0 points determined relative to a baseline level of pain measured immediately before the period T¹ is begun and measured according to pain intensity scores of the Pain Intensity Numeric Rating Scale (PI-NRS).

As defined herein, the obtained pain reduction is a mean value in pain reduction, preferably determined from pain intensity scores of the Pain Intensity Numeric Rating Scale (PI-NRS).

The method is effective in reducing, preferably eliminating, the need of a second pain medication different from the compound according to formula (I). This does not preclude use of other pain medications but as shown in the studies below, less than 50% of patients treated with AP-325 used a secondary medication whereas over 70% of the placebo group used a second “rescue” pain medication by the end of period T². In related embodiments, the method is effective in reducing, preferably eliminating, the need of the second pain medication in greater than 40%, preferably 45% or greater, more preferably 50% or greater of the mean number of patients undergoing the method of treating neuropathic pain.

Pain measured as disturbances in sleep is well known to those skilled in the art. In general embodiments, an absolute improvement in the mean value according to the Daily Sleep Interference Scale (DSIS), determined relative to a baseline level of the mean value according to the Daily Sleep Interference Scale (DSIS) measured immediately before the period T¹ is begun, is obtained concomitant with pain reduction over the duration of period T² of the treatment cycle. In more specific embodiments, absolute improvement in the mean value according to the Daily Sleep Interference Scale (DSIS) is greater than 1.0 point, preferably greater than 1.1 points, preferably greater than 1.2 points, and/or in the range of 1.0 to 5.0 points, more preferably in the range of 2.0 to 4.5 points, more preferably in the range of 2.5 to 4.0 points, more preferably in the range of 2.5 to 3.5 points, more preferably in the range of 2.5 to 3.0 points or 1.0 point to 2 points, more preferably in the range of 1.1 to 1.5 points, determined relative to a baseline level of the mean value according to the Daily Sleep Interference Scale (DSIS) measured immediately before the period T¹ is begun.

In general embodiments, an improvement in the mean value of the Hospital Anxiety and Depression Score (HADS), determined relative to a baseline level of the mean value according to the Hospital Anxiety and Depression Score (HADS) measured immediately before the period T¹ is begun, is obtained concomitant with pain reduction over the duration of period T² of the treatment cycle. In related embodiments, improvement in the mean value of the Hospital Anxiety and Depression Score (HADS) is greater than 0.1 point, preferably greater than 0.2 points, preferably greater than or about equal to 0.5 points, determined relative to a baseline level of the mean value according to the Daily Sleep Interference Scale measured immediately before the period T¹ is begun, is obtained concomitant with pain reduction over the duration of period T² of the treatment cycle.

In general embodiments of the invention, patients undergoing the treatment cycle have a successful treatment determined at the end of the period of T² according to the Patient Global Impression Of Change (PGIC) scale, preferably wherein a treatment is successful if a patient reports a much improved to very much improved overall status according to the Patient Global Impression Of Change (PGIC) scale. In some embodiments, at least 20%, preferably at least 30%, more preferably at least 35%, more preferably at least 40%, more preferably at least 45% of patients undergoing the treatment cycle have a successful treatment determined at the end of the period of T² according to the Patient Global Impression Of Change (PGIC) scale, preferably wherein a treatment is successful if a patient reports a much improved to very much improved overall status according to the Patient Global Impression Of Change (PGIC) scale.

### Reduced Side Effects

The present invention permits a very “clean” side effect profile, similar to placebo as shown below in the examples. Side effects or Adverse Events are necessarily the focus of clinical trials. Thus, in general embodiments according to the present disclosure, less than 30% of patients, preferably less than 25% of patients, undergoing the treatment cycle have had a Treatment Emergent Adverse Event (TEAE) determined at the end of the period of T². Specifically, the Treatment Emergent Adverse Event (TEAE) is chosen from the group consisting of diarrhea, flatulence, nausea, asthenia (lack of energy), fatigue, nasopharyngitis, tonsillitis, blood creatine kinase increased, blood triglycerides increased, back pain, drowsiness, dizziness, somnolence and sedation. In related embodiments, less than 30% of patients, preferably less than 25% of patients, more preferably none of the patients undergoing the treatment cycle have had a Treatment Emergent Adverse Event (TEAE) chosen from the group consisting of drowsiness, dizziness, somnolence and sedation, preferably drowsiness, dizziness and somnolence, determined at the end of the period of T².

Less than 5% of the total dosage of the compound according to formula (I) is believed to enter the brain, preferably calculated as the concentration of the compound in brain to the concentration of the compound in blood. This pharmacological property could explain the improved side effect profile, at least with regards to CNS related adverse events. Since AP-325 does not appreciably enter the CNS, this also suggests that none of the patients undergoing treatment will experience drug withdrawal, drug withdrawal syndrome, or substance withdrawal syndrome, specifically in comparison with substances such as oxycontin.

### Compounds for Use in Treating Neuropathic Pain

The present disclosure also relates compound according to formula (I), or an isomer thereof, or a tautomer thereof, or an isotope thereof; having the structure of:

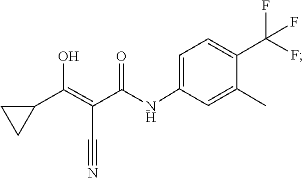 [SMILES: Cc1cc(NC(=O)/C(C#N)=C(\O)C2CC2)ccc1C(F)(F)F]

for use in treating neuropathic pain in a method according to any one of items 1 to 116 and to the embodiments and figures as disclosed herein.

### Use of a Compound for Preparing a Pharmaceutical Composition

The present disclosure also relates the use of a compound according to formula (I), or an isomer thereof, or a tautomer thereof, or an isotope thereof; having the structure of:

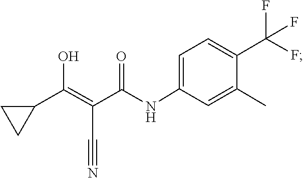 [SMILES: Cc1cc(NC(=O)/C(C#N)=C(\O)C2CC2)ccc1C(F)(F)F]

for preparing a pharmaceutical composition for treating a human patient suffering from neuropathic pain, wherein the use comprises a method according to any one of items 1 to 116 and to the embodiments and figures as disclosed herein.

### ITEMS OF THE INVENTION

The present invention is also characterized by the following items:
1. A method of treating neuropathic pain in human patients comprising
a treatment cycle comprising:
   administering a compound according to formula (I), or an isomer thereof, or a tautomer thereof, or an isotope thereof, over a period T¹; formula (I) having the structure of:

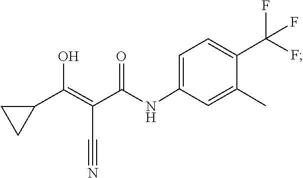 [SMILES: Cc1cc(NC(=O)/C(C#N)=C(\O)C2CC2)ccc1C(F)(F)F]

discontinuing the administration of the compound according to formula (I) for a period T² that immediately follows T¹; —obtaining pain reduction over the total sum of the periods T¹ and T² of the treatment cycle.
2. A method of immune resolution therapy (IRT) comprising
a treatment cycle comprising:
   administering a compound according to formula (I), or an isomer thereof, or a tautomer thereof, or an isotope thereof, over a period T¹; formula (I) having the structure of:

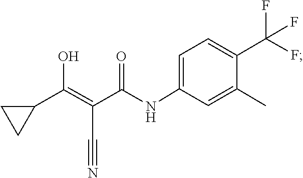 [SMILES: Cc1cc(NC(=O)/C(C#N)=C(\O)C2CC2)ccc1C(F)(F)F]

inhibiting of dihydroorotate dehydrogenase (DHODH) during T¹;
modulating GABA receptors during T¹;
discontinuing the administration of the compound according to formula (I) for a period T² that immediately follows T¹;
clearing the compound according to formula (I) during 12;
modulating lymphocyte subpopulations during T² relative to the last day of T¹, optionally determined by flow cytometry or Fluorescence-Activated Cell Sorting (FACS);
obtaining pain reduction over the total sum of the periods T¹ and T² of the treatment cycle.
3. The method of claim 2, wherein the method of immune resolution therapy (IRT) is for treating neuropathic pain optionally in human patients.
4. The method of any one of the preceding items, wherein during T² modulating lymphocyte subpopulations comprises increasing the Treg lymphocyte population and/or the Breg lymphocyte population relative to the Treg lymphocyte population and/or the Breg lymphocyte population measured at the end of T¹ determined by flow cytometry or FAGS. 5.
   The method of any one of the preceding items, wherein during T² modulating lymphocyte subpopulations comprises increasing the Treg lymphocyte population relative to the Treg lymphocyte population measured at the end of T¹ determined by flow cytometry or FAGS.
6. The method of any one of the preceding items, wherein during T² modulating lymphocyte subpopulations comprises increasing the Breg lymphocyte population relative to the Breg lymphocyte population measured at the end of T¹ determined by flow cytometry or FAGS.
7. The method of any one of the preceding items, wherein during T², modulating lymphocyte subpopulation comprises increasing the Treg lymphocyte population diversity and/or the Breg lymphocyte population diversity relative to the Treg lymphocyte population diversity and/or the Breg lymphocyte population diversity measured at the end of T¹ determined by flow cytometry or FACS. 8. The method of any one of the preceding items, wherein during T², modulating lymphocyte subpopulation comprises increasing the Treg lymphocyte population diversity relative to the Treg lymphocyte population diversity measured at the end of T¹ determined by flow cytometry or FAGS.
9. The method of any one of the preceding items, wherein during T², modulating lymphocyte subpopulation comprises increasing the Breg lymphocyte population diversity relative to the Breg lymphocyte population diversity measured at the end of T¹ determined by flow cytometry or FAGS.
10. The method of any one of the preceding items, wherein beginning T², the compound according to formula I is cleared within 1 to 7 days, preferably within 1 to 3 days, determined by pharmacokinetic analysis using mass-spectroscopy, preferably by pharmacokinetic dose/AUG analysis by means of LC-MS/MS (Liquid Chromatography-Tandem Mass Spectrometry).
11. The method of any one of the preceding items, wherein modulating the lymphocyte subpopulation comprises lowering the Th1 lymphocyte population and/or lowering the Th17 lymphocyte population and/or lowering the inflammatory B lymphocyte (B cells) population, measured at the end of T¹ relative to the Th1 lymphocyte population and/or the Th17 lymphocyte population and/or the inflammatory B lymphocyte (B cells) population measured at the end of T², determined by flow cytometry or FAGS.
15. The method of any one of the preceding items, wherein modulating the lymphocyte subpopulation comprises the Treg lymphocyte population diversity and/or the Breg lymphocyte population diversity measured at the end of T¹ is lower than the Treg lymphocyte population diversity and/or the Breg lymphocyte population diversity measured at the end of T², determined by flow cytometry or FAGS.
16. The method of any one of the preceding items, wherein modulating the lymphocyte subpopulation comprises the Treg lymphocyte population diversity measured at the end of T¹ is lower than the Treg lymphocyte population diversity measured at the end of T², determined by flow cytometry or FAGS.
17. The method of any one of the preceding items, wherein modulating the lymphocyte subpopulation comprises the Breg lymphocyte population diversity measured at the end of T¹ is lower than the Breg lymphocyte population diversity measured at the end of T², determined by flow cytometry or FAGS.
18. The method of any one of the preceding items, wherein the modulating of the lymphocyte subpopulations comprises the Th1 lymphocyte population and/or the Th17 lymphocyte population and/or the inflammatory B lymphocyte (B cells) population, measured at the end of T¹ is lower than the Th1 lymphocyte population and/or the Th17 lymphocyte population and/or the inflammatory B lymphocyte (B cells) population measured as the baseline level immediately before the period T¹ is begun, determined by flow cytometry or FAGS.
19. The method of any one of the preceding items, wherein the modulating the lymphocyte subpopulations comprises the Th1 lymphocyte population measured at the end of T¹ is lower than the Th1 lymphocyte population measured as the baseline level immediately before the period T¹ is begun, determined by flow cytometry or FAGS.
20. The method of any one of the preceding items, wherein the modulating the lymphocyte subpopulations comprises the Th17 lymphocyte population measured at the end of T¹ is lower than the Th17 lymphocyte population measured as the baseline level immediately before the period T¹ is begun, determined by flow cytometry or FAGS.
21. The method of any one of the preceding items, wherein the modulating the lymphocyte subpopulations comprises the inflammatory B lymphocyte (B cells) population, measured at the end of T¹ is lower than the inflammatory B lymphocyte (B cells) population measured as the baseline level immediately before the period T¹ is begun, determined by flow cytometry or FACS. 22. The method of any one of the preceding items, wherein modulating the lymphocyte subpopulation comprises the Treg lymphocyte population diversity and/or the Breg lymphocyte population diversity measured at the end of T¹ is lower than Treg lymphocyte population diversity and/or the Breg lymphocyte population diversity measured as the baseline level immediately before the period T¹ is begun, determined by flow cytometry or FAGS.
23. The method of any one of the preceding items, wherein modulating the lymphocyte subpopulation comprises the Treg lymphocyte population diversity measured at the end of T¹ is lower than the Treg lymphocyte population diversity measured as the baseline level immediately before the period T¹ is begun, determined by flow cytometry or FAGS.
24. The method of any one of the preceding items, wherein modulating the lymphocyte subpopulation comprises the Breg lymphocyte population diversity measured at the end of T¹ is lower than the Breg lymphocyte population diversity measured as the baseline level immediately before the period T¹ is begun, determined by flow cytometry or FAGS.
25. The method of any one of the preceding items, wherein obtaining pain reduction over the total sum of the periods T¹ and T² of the treatment cycle is determined according to pain intensity scores of the Pain Intensity Numeric Rating Scale (PI-NRS).
26. The method of any one of the preceding items, wherein for the duration of T², the obtained pain reduction is about equal to or greater than the maximum pain reduction measured during T¹, preferably determined according to pain intensity scores of the Pain Intensity Numeric Rating Scale (PI-NRS).
27. The method of any one of the preceding items, wherein for the duration of T², the obtained pain reduction is greater than 15%, preferably greater than 20% determined relative to a baseline level of pain measured immediately before the period T¹ is begun, preferably measured according to pain intensity scores of the Pain Intensity Numeric Rating Scale (PI-NRS).
28. The method of item 27, wherein the obtained pain reduction is greater than 25%, preferably greater than 26%.
29. The method of item 27 or 28, wherein the obtained pain reduction is in the range of from 15% to 100%, preferably is in the range of from 20% to 100%, more preferably in the range of from 21% to 90%; more preferably in the range of from 22% to 80%; more preferably in the range of from 23% to 70%; more preferably in the range of from 24% to 60%; more preferably in the range of from 25% to 50%, more preferably in the range of from 25% to 40% more preferably in the range of from 25% to 35% 30. The method of any one of the preceding items, wherein the maximum obtained pain reduction for T¹ is greater than 15%, preferably greater than 20%, more preferably greater than 25%, determined relative to the baseline level of pain measured immediately before the period T¹ is begun, preferably measured according to pain intensity scores of the Pain Intensity Numeric Rating Scale (PI-NRS).
31. The method of item 30, wherein the obtained pain reduction is in the range of from 15% to 100%, preferably is in the range of from 20% to 100%, more preferably in the range of from 21% to 90%, more preferably in the range of from 22% to 80%, more preferably in the range of from 23% to 70%, more preferably in the range of from 24% to 60%, more preferably in the range of from 25% to 50%, more preferably in the range of from 25% to 40% more preferably in the range of from 25% to 35%, more preferably in the range of from 25% to 30% or in the range of from 40% to 45%, more preferably in the range of from 40% to 45%.
32. The method of any one of the preceding items, wherein the absolute pain reduction, preferably the mean absolute pain reduction, over the total sum of the periods T¹ and T² of the treatment cycle is greater than 1.0 point, preferably greater than 1.3 points, preferably greater than 1.5 points, more preferably greater than 2 points, more preferably greater than 2.5 points, more preferably in the range of 1.0 to 5.0 points, more preferably in the range of 2.0 to 4.5 points, more preferably in the range of 2.5 to 4.0 points, more preferably in the range of 2.5 to 3.5 points, more preferably in the range of 2.5 to 3.0 points determined relative to a baseline level of pain measured immediately before the period T¹ is begun and measured according to pain intensity scores of the Pain Intensity Numeric Rating Scale (PI-NRS).
33. The method of any one of the preceding items, wherein the obtained pain reduction is a mean value in pain reduction, preferably determined from pain intensity scores of the Pain Intensity Numeric Rating Scale (PI-NRS).
34. The method of any one of the preceding items, wherein the method is effective in reducing, preferably eliminating, the need of a second pain medication different from the compound according to formula (I).
35. The method of item 34, wherein the method is effective in reducing, preferably eliminating, the need of the second pain medication in greater than 40%, preferably 45% or greater, more preferably 50% or greater of the mean number of patients undergoing the method of treating neuropathic pain.
36. The method of any one of the preceding items, wherein an absolute improvement in the mean value according to the Daily Sleep Interference Scale (DSIS), determined relative to a baseline level of the mean value according to the Daily Sleep Interference Scale (DSIS) measured immediately before the period T¹ is begun, is obtained concomitant with pain reduction over the duration of period T² of the treatment cycle.
37. The method of item 36, wherein absolute improvement in the mean value according to the Daily Sleep Interference Scale (DSIS) is greater than 1.0 point, preferably greater than 1.1 points, preferably greater than 1.2 points, and/or in the range of 1.0 to 5.0 points, more preferably in the range of 2.0 to 4.5 points, more preferably in the range of 2.5 to 4.0 points, more preferably in the range of 2.5 to 3.5 points, more preferably in the range of 2.5 to 3.0 points and/or optionally 1.0 point to 2 points, more preferably in the range of 1.1 to 1.5 points, determined relative to a baseline level of the mean value according to the Daily Sleep Interference Scale (DSIS) measured immediately before the period T¹ is begun.
36. The method of any one of the preceding items, wherein an improvement in the mean value of the Hospital Anxiety and Depression Score (HADS), determined relative to a baseline level of the mean value according to the Hospital Anxiety and Depression Score (HADS) measured immediately before the period T¹ is begun, is obtained concomitant with pain reduction over the duration of period T² of the treatment cycle.
37. The method of item 36, wherein improvement in the mean value of the Hospital Anxiety and Depression Score (HADS) is greater than 0.1 point, preferably greater than 0.2 points, preferably greater than or about equal to 0.5 points, determined relative to a baseline level of the mean value according to the Daily Sleep Interference Scale measured immediately before the period T¹ is begun, is obtained concomitant with pain reduction over the duration of period T² of the treatment cycle.
38. The method of any one of the preceding items, wherein patients undergoing the treatment cycle have a successful treatment determined at the end of the period of T² according to the Patient Global Impression of Change (PGIC) scale, preferably wherein a treatment is successful if a patient reports a much improved to very much improved overall status according to the Patient Global Impression Of Change (PGIC) scale.
39. The method of any one of the preceding items, wherein at least 20%, preferably at least 30%, preferably at least 35%, more preferably at least 40% of patients undergoing the treatment cycle have a successful treatment determined at the end of the period of T² according to the Patient Global Impression of Change (PGIC) scale, preferably wherein a treatment is successful if a patient reports a much improved to very much improved overall status according to the Patient Global Impression Of Change (PGIC) scale.
40. The method of any one of the preceding items, wherein less than 30% of patients, preferably less than 25% of patients, undergoing the treatment cycle have had a Treatment Emergent Adverse Event (TEAE) determined at the end of the period of T². 41. The method of item 40, wherein the Treatment Emergent Adverse Event (TEAE) is chosen from the group consisting of diarrhea, flatulence, nausea, asthenia (lack of energy), fatigue, nasopharyngitis, tonsillitis, blood creatine kinase increased, blood triglycerides increased, back pain, drowsiness, dizziness, somnolence and sedation.
42. The method of any one of the preceding items, preferably item 32 or 33, wherein less than 30% of patients, preferably less than 25% of patients, more preferably none of the patients undergoing the treatment cycle have had a Treatment Emergent Adverse Event (TEAE) chosen from the group consisting of drowsiness, dizziness, somnolence and sedation, preferably drowsiness, dizziness and somnolence, determined at the end of the period of T².
431. The method of any one of the preceding items, wherein period T²≥period T¹, preferably period T²≥2×period T¹.
44. The method of any one of the preceding items, wherein period T¹≤20 days, preferably period T¹≤17 days, more preferably period T¹≤15 days.
45. The method of any one of the preceding items, wherein period T¹ is in the range of from 1 to 100 days, preferably 1 to 84 days, more preferably 1 to 42 days, more preferably 1 to 28 days, more preferably 1 to 21 days, more preferably in the range of from 2 to 19 days, more preferably in the range of from 3 to 18 days, more preferably in the range of from 4 to 17 days, more preferably in the range of from 5 to 16 days, more preferably in the range of from 6 to 15 days, more preferably in the range of from 7 to 14 days, more preferably in the range of from 8 to 13 days, more preferably in the range of from 9 to 12 days, more preferably in the range of from 10 to 11 days.
46. The method of any one of the preceding items, wherein period T²≥1 day, preferably period T²≥2 days, more preferably period T²≥3 days, more preferably period T²≥4 days, more preferably period T²≥5 days, more preferably period T²≥10 days, more preferably period T²≥15 days, more preferably period T²≥17 days, more preferably period T²≥20 days, more preferably period T²≥21 days, more preferably period T²≥28 days, more preferably period T²≥30 days, more preferably period T²≥34 days, more preferably period T²≥40 days, more preferably period T²≥42 days, more preferably period T²≥56 days more preferably period T²≥84 days.
47. The method of any one of the preceding items, wherein period T² is in the range of from 1 to 200 days, preferably in the range of from 1 to 168 days, more preferably in the range of from 1 to 100 days, preferably in the range of from 1 to 84 days, preferably in the range of from 1 to 56 days, preferably in the range of from 1 to 50 days, preferably in the range of from 1 to 42 days, more preferably in the range of from 1 to 40 days, more preferably in the range of from 4 to 38 days, more preferably in the range of from 6 to 36 days, more preferably in the range of from 8 to 34 days, more preferably in the range of from 10 to 32 days, more preferably in the range of from 12 to 30 days, more preferably in the range of from 14 to 28 days, more preferably in the range of from 16 to 26 days, more preferably in the range of from 18 to 24 days, more preferably in the range of from 20 to 22 days.
48. The method of any one of the preceding items, wherein the administering of the patient with the compound according to formula (I) over a period T¹ is with an effective amount.
48. The method of any one of the preceding items, wherein the administering of the patient with the compound according to formula (I) over a period T¹ is with an amount, preferably an effective amount according to item 36, in the range of from 0.05 mg of the compound per 1 kg of the patient to 5.0 mg of the compound per 1 kg of the patient, preferably 0.1 mg of the compound per 1 kg of the patient to 5.0 mg of the compound per 1 kg of the patient, more preferably in the range of from 0.25 mg of the compound per 1 kg of the patient to 3.0 mg of the compound per 1 kg of the patient, more preferably in the range of from in 0.7 mg of the compound per 1 kg of the patient to 2.7 mg of the compound per 1 kg of the patient, more preferably in the range of from in 0.9 mg of the compound per 1 kg of the patient to 2.5 mg of the compound per 1 kg of the patient, more preferably in the range of from in 1.0 mg of the compound per 1 kg of the patient to 2.0 mg of the compound per 1 kg of the patient.
49. The method of any one of the preceding items, wherein the patient is dosed daily with a compound according to formula (I) over the period T¹.
50. The method of item 49, wherein the compound according to formula (I) is administered once per day.
51. The method of item 49, wherein the compound according to formula (I) is administered one or more times per day, preferably two to three times per day.
52. The method of any one of the preceding items, wherein the compound according to formula (I) is administered orally.
53. The method of any one of the preceding items, wherein the compound according to formula (I) is administered in the form of a capsule, tablet, granules, drop or liquid.
54. The method of any one of the preceding items, wherein the compound according to formula (I) is administered in the form of a capsule or tablet.
55. The method of item 54, wherein an individual capsule or tablet comprises the compound according to formula (I) in an amount in the range of from 2.5 to 250 mg, preferably 2.5 to 100 mg, more preferably 5 to 50 mg, more preferably 10 to 30 mg, preferably in the range of from 20 to 30 mg, more preferably being about 25 mg or more preferably about 100 mg.
56. The method of any one of the preceding items, wherein the compound according to formula (I) is administered as a pharmaceutical composition.
57. The method of item 56, wherein the pharmaceutical composition comprises one or more excipients, preferably one or more pharmaceutically acceptable excipients.
58. The method of item 57, wherein the one or more excipients are selected from the list consisting of solvent, binders, surfactants, stabilizers, fillers, disintegrants, lubricants, bulking agents, anti-caking agents, glidants, preservatives and buffering agents, preferably selected from the group consisting of fillers, binders, glidants, lubricants, and disintegrants.
59. The method of item 57 or 58, wherein the excipients comprise, preferably consists of, poloxamer 407, sodium glycocholate, hypromellose (hydroxypropylmethylcellulose), isomalt and talc.
60. The method of any one of the preceding items, wherein the compound according to formula (I) is in the form of a salt, preferably a pharmaceutically acceptable salt, more preferably in the form of a sodium salt.
61. The method of any one of the preceding items, wherein the neuropathic pain is peripheral and/or predominantly peripheral neuropathic pain or central and/or predominantly central neuropathic pain.
62. The method of any one of the preceding items, wherein the neuropathic pain is peripheral neuropathy caused by damage to peripheral nerves resulting in symptoms chosen from the group consisting of numbness, tingling, autonomic dysfunction, pain, and weakness in the extremities.
63. The method of any one of the preceding items, wherein the neuropathic pain is peripheral neuropathy caused by axonal degeneration.
64. The method of item 63, wherein the peripheral neuropathy caused by axonal degeneration is characterized by deterioration or death of the long projections of neurons (axons).
65. The method of item 63 or 64, wherein the axonal degeneration is resultant from metabolic dysfunction, oxidative stress, mitochondrial impairment and/or disruption of axonal transport.
66. The method of any one of the preceding items, wherein the neuropathic pain is peripheral neuropathy chosen from the group of causations consisting of toxins (optionally Drug-Induced Peripheral Neuropathy (DIPN) preferably resulting from alcohol and/or drug abuse), inflammation/immune-mediated, hereditary, vasculitic disease, amyloid neuropathy, sarcoidosis, connective tissue disease, thyroid dysfunction, vitamin B12 deficiency and small fiber neuropathy.
67. The method of any one of the preceding items, wherein the neuropathic pain is resultant from or is characterized by a cause that is selected from the group of systemic diseases, e.g. diabetic neuropathy; drug-induced lesions, e.g. neuropathy due to chemotherapy; traumatic syndrome and entrapment syndrome; lesions in nerve roots and posterior ganglia; neuropathies after HIV infections; neuralgia after Herpes infections; nerve root avulsions; cranial nerve lesions; cranial neuralgias, e.g., trigeminal neuralgia; neuropathic cancer pain; phantom pain; compression of peripheral nerves, neuroplexus and nerve roots; paraneoplastic peripheral neuropathy and ganglionopathy; complications of cancer therapies, e.g. chemotherapy, irradiation, and surgical interventions; complex regional pain syndrome; type I lesions (previously known as sympathetic reflex dystrophy]; and type II lesions (corresponding approximately to causalgia]; cerebral lesions that are predominantly thalamic; infarction, e.g. thalamic infarction or brain stem infarction; cerebral tumors or abscesses compressing the thalamus or brain stem; multiple sclerosis; brain operations, e.g. thalamotomy in cases of motoric disorders; spinal cord lesions; spinal cord injuries; spinal cord operations, e.g. anterolateral cordotomy; ischemic lesions; anterior spinal artery syndrome; Wallenberg's syndrome; and syringomyelia.
68. The method of any one of the preceding items, wherein the neuropathic pain is postherpetic neuralgia (caused by Herpes Zoster], root avulsions, painful traumatic mononeuropathy, painful polyneuropathy (particularly due to diabetes], central pain syndromes (potentially caused by virtually any lesion at any level of the nervous system], postsurgical pain syndromes (eg, postmastectomy syndrome, post thoracotomy syndrome, phantom pain] and/or complex regional pain syndrome (reflex sympathetic dystrophy and causalgia].
69. The method of any one of the preceding items, wherein the neuropathic pain is a central pain syndrome caused by spinal cord injury and/or spinal cord contusion.
70. The method of any one of the preceding items, wherein the neuropathic pain is idiopathic
71. The method of any one of the preceding items, wherein the neuropathic pain is a chronic neuropathic pain, preferably wherein the chronic neuropathic pain is early chronic neuropathic pain characterized by persistent pain existing for a duration in the range of 4 to 12 months or the chronic neuropathic pain is persistent pain existing for a duration of 1 to 15 years.
72. The method of any one of the preceding items, wherein the neuropathic pain is post surgical neuropathic pain, preferably peripheral post-surgical neuropathic pain.
73. The method of any one of the preceding items, wherein the neuropathic pain is post-surgical neuropathic pain, preferably peripheral post-surgical neuropathic pain selected from the group consisting of breast surgery, chest surgery, hernia repair, abdominal surgery, varicose vein surgery or gynecologic surgery.
74. The method of any one of the preceding items, wherein the neuropathic pain is post surgical neuropathic pain after breast surgery.
75. The method of any one of the preceding items, wherein the neuropathic pain is post surgical neuropathic pain after chest surgery.
76. The method of any one of the preceding items, wherein the neuropathic pain is post surgical neuropathic pain after hernia repair.
77. The method of any one of the preceding items, wherein the neuropathic pain is post surgical neuropathic pain after abdominal surgery.
78. The method of any one of the preceding items, wherein the neuropathic pain is post surgical neuropathic pain after varicose vein surgery.
79. The method of any one of the preceding items, wherein the neuropathic pain is post surgical neuropathic pain after gynecologic surgery.
80. The method of any one of the preceding items, wherein the patients are 18 years of age or older.
81. The method of any one of the preceding items, wherein the treatment cycle is repeated, preferably as necessary for management of recurring neuropathic pain or chronic neuropathic pain.
82. The method of any one of the preceding items, wherein only negligible amounts (of 10% or less, preferably 10% to >1%), preferably trace amounts (preferably 1% or less, more preferably 0.1% or less, more preferably 0.05% or less), more preferably no detectable amount, of the compound according to formula (I) is detectable within the patients plasma on the final day of T², preferably determined by HPLC/mass spectroscopy.
83. The method of any one of the preceding items, wherein only negligible, preferably trace amounts, more preferably no detectable amount, of metabolites of the compound according to formula (I) are detectable within the patients plasma on the final day of T².
81. The method of any one of the preceding items, wherein no detectable amount of the compound according to formula (I) is detectable within the patients plasma on the final day of T².
85. The method of any one of the preceding items, wherein less than 5% of the total dosage of the compound according to formula (I) enters the brain, preferably calculated as the concentration of the compound in brain to the concentration of the compound in blood.
86. The method of any one of the preceding items, wherein the method comprises one or more additional treatment cycles.
87. The method of any one of the preceding items, wherein the method comprises an evaluation period T^{Ev} that immediately follows period T².
88. The method of item 87, wherein the evaluation period T^{Ev} is equal to the number of days required for the loss in absolute pain reduction, preferably the mean absolute pain reduction, to be greater than 1.0 point, preferably greater than 1.3 points, preferably greater than 1.5 points, determined relative to the obtained absolute pain reduction, preferably the obtained mean absolute pain reduction, measured on the final day of period T² and determined according to pain intensity scores of the Pain Intensity Numeric Rating Scale (PI-NRS).
89. The method of item 87 or 88, wherein a further treatment cycle is initiated immediately following T^{EV}, preferably the further treatment cycle is initiated when the loss in absolute pain reduction, preferably the mean absolute pain reduction, is greater than 1.0 point, preferably greater than 1.3 points, preferably greater than 1.5 points, determined relative to the obtained absolute pain reduction, preferably the obtained mean absolute pain reduction, measured on the final day of period T² and determined according to pain intensity scores of the Pain Intensity Numeric Rating Scale (PI-NRS).
90. The method of any one of items 87 to 89, wherein the evaluation period T^{Ev} is equal to the number of days required for the loss in pain reduction, preferably the loss in mean pain reduction, is greater than 15%, preferably greater than 20% determined relative to the obtained pain reduction, preferably the obtained mean pain reduction, measured on the final day of period T² and determined according to pain intensity scores of the Pain Intensity Numeric Rating Scale (PI-NRS).
91. The method of any one of items 87 to 90, wherein a further treatment cycle is initiated immediately following T^{EV}, preferably the further treatment cycle is initiated when the loss in pain reduction is greater than 15%, preferably greater than 20% determined relative to the obtained pain reduction, preferably the obtained mean pain reduction, measured on the final day of period T² and determined according to pain intensity scores of the Pain Intensity Numeric Rating Scale (PI-NRS).
92. The method of any one of the preceding items, wherein none of the patients undergoing treatment experience drug withdrawal, drug withdrawal syndrome, or substance withdrawal syndrome.
93. The method of anyone of the preceding items, wherein the treatment period T1 is about 14 days and the drug-free period T2 is about 14 days.
94. The method of anyone of the preceding items, wherein the treatment period T1 is about 28 days and the drug-free period T2 is about 28 days.
95. The method of anyone of the preceding items, wherein the treatment period T1 is about 21 days and the drug-free period T2 is about 21 days.
96. The method of anyone of the preceding items, wherein the treatment period T1 is about 14 days and the drug-free period T2 is about 28 days.
97. The method of anyone of the preceding items, wherein the treatment period T1 is about 28 days and the drug-free period T2 is about 56 days.
98. The method of anyone of the preceding items, wherein the treatment period T1 is about 21 days and the drug-free period T2 is about 42 days.
99. The method of anyone of the preceding items, wherein the treatment period T1 is about 10 days and the drug-free period T2 is about 30 days.
100. The method of anyone of the preceding items, wherein the treatment period T1 is about 14 days and the drug-free period T2 is about 56 days.
101. The method of anyone of the preceding items, wherein the treatment period T1 is about 42 days and the drug-free period T2 is about 84 days.
102. The method of anyone of the preceding items, wherein the treatment period T1 is about 56 days and the drug-free period T2 is about 56 days.
103. The method of anyone of the preceding items, wherein the treatment period T1 is about 84 days and the drug-free period T2 is about 84 days.
104. The method of anyone of the preceding items, wherein the treatment period T1 is about 28 days and the drug-free period T2 is about 84 days.
105. The method of anyone of the preceding items, wherein the treatment period T1 is about 21 days and the drug-free period T2 is about 90 days.
106. The method of anyone of the preceding items, wherein the treatment period T1 is about 14 days and the drug-free period T2 is about 120 days.
107. The method of anyone of the preceding items, wherein the treatment period T1 is about 56 days and the drug-free period T2 is about 168 days.
108. The method of anyone of the preceding items, wherein the treatment period T1 is about 84 days and the drug-free period T2 is about 168 days.
109. The method of any one of the preceding items, wherein the population of pro-inflammatory CD4+ T cells that secrete IFN-γ and IL-17 measured at the end of T¹ is lower than the population of pro-inflammatory CD4+ T cells that secrete IFN-γ and IL-17 measured as the baseline level immediately before the period T¹ is begun, determined by flow cytometry or FAGS.
110. The method of item 109, wherein the population of pro-inflammatory CD4+ T cells that secrete IFN-γ and IL-17 are Th1/Th17 cells.
111. The method of any one of the preceding items, wherein the population of B cell clones producing autoreactive IgG against neuronal antigens measured at the end of T¹ is lower than the population of B cell clones producing autoreactive IgG against neuronal antigens measured as the baseline level immediately before the period T¹ is begun, determined by flow cytometry or FAGS.
112. The method of any one of the preceding items, wherein the concentration of any one of TNF-α, IL-1β, IL-6, and/or IL-17 measured at the end of T¹ is lower than the concentration of TNF-α, IL-1β, IL-6, and/or IL-17 measured as the baseline level immediately before the period T¹ is begun, determined by immunoassay, preferably by ELISA or by using multiplex bead based immunoassay specific for human cytokines.
113. The method of any one of the preceding items, wherein the last day of T², the ratio of the population of regulatory T cells (Tregs) to the population of effector T cells is higher than said ratio measured at the end of T¹ determined by flow cytometry or FAGS.
114. The method of any one of the preceding items, wherein the last day of T², the concentration of IL-2 increased relative to the concentration measured at the end of T¹, determined by determined by immunoassay, preferably by ELISA or by using multiplex bead based immunoassay specific for human cytokines.
115. The method of any one of the preceding items, wherein the last day of T², the concentration of FoxP3, CTLA-4, IL-10 and/or TGF-β increased relative to the concentration measured at the end of T¹, determined by determined by immunoassay, preferably by ELISA or by using multiplex bead based immunoassay specific for human cytokines.
116. The method of any one of the preceding items, wherein the last day of T², the ratio of the population of macrophages in the M2 tissue remodelling, anti-inflammatory phase to the population of macrophages in the M1 pro-inflammatory phase is higher than said ratio measured at the end of T¹ determined by flow cytometry or FAGS.
117. A method of treating neuropathic pain in human patients comprising
a treatment cycle comprising:
   administering a compound according to formula (I) over a period T¹; formula (I)
having the structure of:

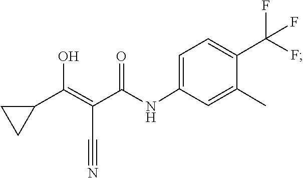 [SMILES: Cc1cc(NC(=O)/C(C#N)=C(\O)C2CC2)ccc1C(F)(F)F]

discontinuing the administration of the compound according to formula (I) for a period T² that immediately follows T¹;
wherein period T²≥period T¹ or period T²≥2×period T¹;
   obtaining pain reduction over the total sum of the periods T¹ and T² of the treatment cycle;
wherein for the duration of T², the obtained pain reduction is about equal to or greater than the maximum pain reduction measured during T¹.
118. A method of treating neuropathic pain in human patients comprising
a treatment cycle comprising:
   administering a compound according to formula (I) over a period T¹; formula (I) having the structure of:

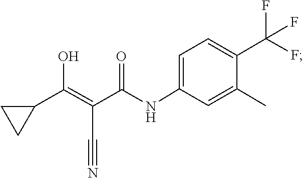 [SMILES: Cc1cc(NC(=O)/C(C#N)=C(\O)C2CC2)ccc1C(F)(F)F]

discontinuing the administration of the compound according to formula (I) for a period T² that immediately follows T¹;
wherein period T²≥period T¹ or period T²≥2×period T¹;
   obtaining pain reduction over the total sum of the periods T¹ and T² of the treatment cycle;
wherein for the duration of T², the obtained pain reduction is about equal to or greater than the maximum pain reduction measured during T¹;
wherein at least 20%, at least 30% or at least 35% of patients undergoing the treatment cycle have a successful treatment determined at the end of the period of T² according to the Patient Global Impression Of Change (PGIC) scale.
119. A method of treating neuropathic pain in human patients comprising
a treatment cycle comprising:
   administering a compound according to formula (I) over a period T¹; formula (I) having the structure of:

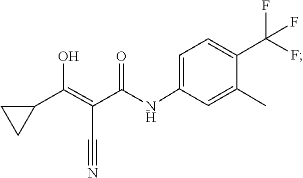 [SMILES: Cc1cc(NC(=O)/C(C#N)=C(\O)C2CC2)ccc1C(F)(F)F]

discontinuing the administration of the compound according to formula (I) for a period T² that immediately follows T¹;
wherein period T²≥period T¹ or period T²≥2×period T¹;
   obtaining pain reduction over the total sum of the periods T¹ and T² of the treatment cycle;
wherein for the duration of T², the obtained pain reduction is about equal to or greater than the maximum pain reduction measured during T¹;
wherein an improvement in the mean value of the Hospital Anxiety and Depression Score (HADS), determined relative to a baseline level of the mean value according to the Hospital Anxiety and Depression Score (HADS) measured immediately before the period T¹ is begun, is obtained concomitant with pain reduction over the duration of period T² of the treatment cycle.
120. A method of treating neuropathic pain in human patients comprising
a treatment cycle comprising:
   administering a compound according to formula (I) over a period T¹; formula (I) having the structure of:

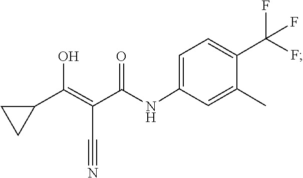 [SMILES: Cc1cc(NC(=O)/C(C#N)=C(\O)C2CC2)ccc1C(F)(F)F]

discontinuing the administration of the compound according to formula (I) for a period T² that immediately follows T¹;
wherein period T²≥period T¹ or period T²≥2×period T¹;
   obtaining pain reduction over the total sum of the periods T¹ and T² of the treatment cycle;
wherein for the duration of T², the obtained pain reduction is about equal to or greater than the maximum pain reduction measured during T¹;
wherein an absolute improvement in the mean value according to the Daily Sleep Interference Scale (DSIS), determined relative to a baseline level of the mean value according to the Daily Sleep Interference Scale (DSIS) measured immediately before the period T¹ is begun, is obtained concomitant with pain reduction over the duration of period T² of the treatment cycle;
wherein an improvement in the mean value of the Hospital Anxiety and Depression Score (HADS), determined relative to a baseline level of the mean value according to the Hospital Anxiety and Depression Score (HADS) measured immediately before the period T¹ is begun, is obtained concomitant with pain reduction over the duration of period T² of the treatment cycle;
wherein patients undergoing the treatment cycle have a successful treatment determined at the end of the period of T² according to the Patient Global Impression Of Change (PGIC) scale, wherein a treatment is successful if a patient reports a much improved to very much improved overall status according to the Patient Global Impression Of Change (PGIC) scale;
wherein less than 30% of patients undergoing the treatment cycle have had a Treatment Emergent Adverse Event (TEAE) determined at the end of the period of 12.
121. A method of treating neuropathic pain in human patients comprising
a treatment cycle comprising:
   administering a compound according to formula (I) over a period T¹; formula (I) having the structure of:

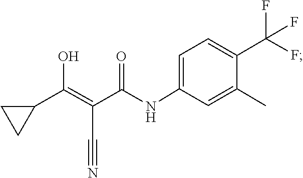 [SMILES: Cc1cc(NC(=O)/C(C#N)=C(\O)C2CC2)ccc1C(F)(F)F]

discontinuing the administration of the compound according to formula (I) for a period T² that immediately follows T¹;
wherein period T²≥period T¹;
   obtaining pain reduction over the total sum of the periods T¹ and T² of the treatment cycle;
wherein for the duration of T², the obtained pain reduction is about equal to or greater than the maximum pain reduction measured during T¹;
wherein none of the patients undergoing treatment experience drug withdrawal, drug withdrawal syndrome, or substance withdrawal syndrome.
122. The method item of any one of items 117 to 121, further comprising the subject-matter of any one of items 1 to 116.
123. A compound according to formula (I), or an isomer thereof, or a tautomer thereof, or an isotope thereof; having the structure of:

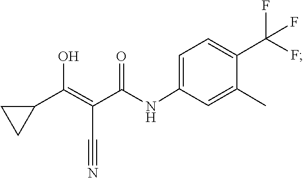 [SMILES: Cc1cc(NC(=O)/C(C#N)=C(\O)C2CC2)ccc1C(F)(F)F]

for use in treating neuropathic pain in a method according to any one of items 1 to 116.
124. Use of a compound according to formula (I), or an isomer thereof, or a tautomer thereof, or an isotope thereof; having the structure of:

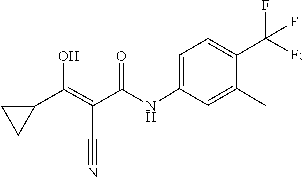 [SMILES: Cc1cc(NC(=O)/C(C#N)=C(\O)C2CC2)ccc1C(F)(F)F]

for preparing a pharmaceutical composition for treating a human patient suffering from neuropathic pain, wherein the use comprises a method according to any one of items 1 to 116.
125. The method, compound or use according to any one of the preceding items, wherein the total daily dosage is in the range of 1 mg to 500 mg per patient, preferably in the range of 25 mg to 250 mg per patient, more preferably in the range of from 50 mg to 150 mg per patient, preferably being about 100 mg per patient.
126. The method of any one of the preceding items, wherein the total daily dosage is in the range of 1 mg to 500 mg per patient, preferably in the range of 25 mg to 250 mg per patient, more preferably in the range of from 50 mg to 150 mg per patient, preferably being about 100 mg per patient.
127. The method of any one of the preceding items, wherein the method comprises modulating lymphocyte subpopulations during T² relative to the last day of T¹, preferably determined by flow cytometry or Fluorescence-Activated Cell Sorting (FACS), preferably determined by flow cytometry.
128. The method of any one of the preceding items, wherein the measurement of a given population of cells is determined by flow cytometry.
129. The method of any one of the preceding items, wherein the measurement of a given population of cells is determined by Fluorescence-Activated Cell Sorting (FACS).

### Examples

### Phase II Clinical Trial

A Phase II clinical trial with repeat oral dosing of AP-325 in adult subjects with Peripheral Post-surgical Neuropathic Pain (PPNP) was performed.

### Methods/Design of the Clinical Trial

This was a Phase II randomized, double-blind, placebo controlled, parallel group study to evaluate the efficacy and safety of AP-325 in subjects with PPNP.

Eligible subjects had undergone a 2-week run-in period consisting of a washout-period of prohibited medications in the 1st week and a baseline period in the 2nd week. If subjects had at least 5 self-reported pain assessments in the baseline period (documented in a diary) and met the required pain criteria, they were randomized to AP-325 or placebo in a 1:1 ratio.

Subjects have taken the IMP (AP-325 or placebo) for 10 days (double-blind treatment period; Days 1-10) and then be followed up for a further 25 days (drug-free period; Days 11-35). An end of study visit was performed on Day 36.

### Eligibility

Subjects between 18 and 80 years of age with a diagnosis of chronic post surgical neuropathic pain after breast surgery (e.g. breast-conserving surgery, mastectomy, surgery to remove lymph nodes), chest surgery (e.g. thoracotomy, video assisted thoracoscopy and sternotomy), hernia repair of the abdominal wall (e.g. femoral hernia repairs, inguinal hernia repairs, umbilical hernia repair or incisional hernia repair), abdominal surgery (e.g. cholecystectomy, appendectomy), varicose vein surgery or gynecologic surgery (e.g. hysterectomy, C-section) were enrolled.

The chronic post-surgical pain had to be developed or increased in intensity after the surgical procedure and persisted beyond the healing process, i.e. at least 3 months after the initiating event, as defined according to the international association for the study of pain (IASP) classification of chronic pain for ICD-11 (Schug et al., 2019).

Subjects must have had ‘probable’ or ‘definite’ neuropathic pain as assessed by the revised IASP special interest group on neuropathic pain (NeuPSIG) grading system (Finnerup et al., 2016).

Key exclusion criteria were for subjects with neuropathic pain not a result of a surgical procedure and any other coexisting pain that could not be discriminated from post surgical neuropathic pain.

### Randomization and Blinding

Key randomization criteria for all subjects were at least 5 daily pain assessments in the baseline week prior to randomization, with a mean score on the PI NRS≥4 and ≤9. Differences between the baseline daily pain scores on the PI-NRS must be ≤50%. For female subjects of childbearing potential, the criterion was a negative pregnancy test in urine on Day 1.

Eligible subjects were randomly assigned in a 1:1 ratio to AP-325 or placebo by means of a computer-generated randomization list.

During the entire study, study participants, investigators, the sponsor, and all other persons involved in the conduct of the study were blinded to treatment. To maintain the blind, AP-325 and placebo capsules had identical appearance, shape and color, and had identical labeling and packaging.

The investigation on futility, safety, and efficacy and the sample size re-estimation at interim analysis have been done by an IDMC consisting of independent clinical and statistical experts. Only the IDMC and an external statistical advisor for supporting the sample size re-estimation had access to unblinded data.

### Sample Size Estimation and Number of Subjects

For the sample size calculation, a 1-sided t-test was considered. Assuming a 1-sided overall significance level of 0.1 and a common standard deviation of 1.8 of the mean difference on PI-NRS at Day 10 and taking the adaptive group-sequential design with one interim analysis (information rate of 50%, O'Brien and Fleming shaped boundaries) into account, a total of 96 subjects (48 for each treatment) had to be sufficient to reveal a mean difference on the PI-NRS of 0.8 with a power of 80% between the treatment and placebo group at the final analysis.

### Study Objectives and Endpoints

The primary objective for this study was to investigate the efficacy of repeat oral dosing of AP-325 on neuropathic pain in subjects with PPNP after 10 days of treatment. The primary endpoint of the study was the change from baseline to Day 10 in the 5-day average pain intensity score based on the Pain Intensity Numerical Rating Scale (PI-NRS).

Secondary objective of efficacy was to further investigate the long-lasting efficacy of repeat oral dosing of AP-325 on neuropathic pain over the entire study duration with the following endpoints:
Longitudinal analysis of the 5-day average PI-NRS score over time from Baseline until Day 35.
Changes from Baseline in the 5-day average PI-NRS score (from Baseline to Day 5, 15, 20, 25, 30 and 35).
Responder rate: proportion of subjects who have a ≥30% reduction in the 5-day average PI-NRS score relative to Baseline (on Days 5, 10, 15, 25 and 35).
Responder rate: proportion of subjects who have a ≥50% reduction in the 5-day average PI-NRS score relative to Baseline (on Days 5, 10, 15, 25 and 35).
Responder rate: proportion of subjects who have a ≥70% reduction in the 5-day average PI-NRS score relative to Baseline (on Days 5, 10, 15, 25 and 35).

A further secondary objective of efficacy was to investigate the effect of repeat oral dosing of AP-325 on patient-reported outcomes with the following endpoints:
Proportion of subjects who “much improved” or “very much improved” relative to Baseline on the patient global impression of change (PGIC) on Days 3, 10, 15, and 36.
Changes from Baseline in the neuropathic pain evaluation using the neuropathic pain symptom inventory (NPSI) questionnaire on Days 3, 10, 15, and 36.
Changes from Baseline in the 5-day average daily sleep interference scale (DSIS) score (from Baseline to Day 5, 10, 15, 25 and 35).
Changes from Baseline in the anxiety and depression assessment using the hospital anxiety and depression scale (HADS) on Days 10 and 36.

One additional secondary objective of efficacy was to investigate the effect of repeat oral dosing of AP-325 on the use of rescue medication with the following endpoint:
Time to first use of rescue medication after randomization.
Total amount of rescue medication used (in mg per day) after randomization.

A secondary objective of safety was to investigate the safety and tolerability of repeat oral dosing of AP-325 in subjects with PPNP with the following endpoints:
Incidence, severity and seriousness of treatment-emergent adverse events (TEAEs).
Changes from Baseline in physical examination and vital signs.
Changes from Baseline in safety laboratory.
Changes from Baseline in 12-lead electrocardiogram.
Changes from Baseline in body weight.

### Statistical Analyses

The hypothesis of a greater change from Baseline to Day 10 in the 5-day average PI-NRS score in the AP-325 group compared to the placebo group was considered with a hypothesis test derived from a linear regression (analysis of covariance [ANCOVA]), corrected for the baseline PI-NRS average pain score. Normality of data could not be assumed if the visual check of the QQ-plot shows: plotted points do not approximately lie on the diagonal line y=x. In case data were clearly not normally distributed, the Wilcoxon Rank Sum test was used to compare the change from Baseline to Day 10 in the 5 day average PI-NRS score in the AP-325 group to the placebo group. A one-sided test procedure was applied at interim analysis using a significance level of 0.0288 and at final analysis using a significance level of 0.0896. For final analysis, the p-values from both analysis stages were combined using the inverse normal combination test with weights based on the initially assumed sample sizes.

Baseline scores were calculated by averaging scores from Days −7 to −1; a minimum of 5 score assessments had to be available. The 5-day average scores were calculated for the time points Day 10, 15, 20, 25, 30, and 35 by averaging the scores from the previous 5 days, where at least 4 assessments had to be available. For example, the Day 10 5-day average score was calculated as the mean of the Day 6-10 scores. At time point Day 5 a 4-day average score was calculated by averaging scores from the previous 4 days, where at least 3 assessments had to be available, e.g. the 4-day average score on Day 5 was calculated as the mean of the Day 2-5 scores.

To assess the difference in the PI-NRS daily pain score between treatments over time a linear mixed-effects model was used (longitudinal analysis). The 4 or 5-day average PI-NRS scores were calculated for Day 5, 15, 20, 25, 30 and 35 and the changes from Baseline were analyzed (cross-sectional analysis) using a mixed-model repeated measures without any imputation for missing data and has an inherent mechanism of imputation under the assumption of missings at random, corrected for the baseline 5-day average PI-NRS score. The responder rate, i.e. the number of subjects with an at least 50% or 30% decrease in the 4 or 5 day average PI NRS score from Baseline were compared between treatments using the Pearson's chi square test.

The PGIC was dichotomized into treatment success (i.e. scoring ‘much improved’ or ‘very much improved’). The hypothesis of a difference in proportions of subjects reporting treatment success on the PGIC between treatments was considered using the Pearson's chi-square test.

Differences in scores of the NPSI, DSIS, and HADS were compared between treatments using linear regression (ANCOVA), corrected for the baseline value, if applicable. For the DSIS, 4 or 5-day averages were calculated similar to the PI-NRS.

Time to first use of rescue medication was analyzed using Kaplan-Meier methods. The total amount of rescue medication (i.e. the mg of rescue medication per day) was tabulated.

All other study endpoints were analyzed descriptively.

### Interims Analysis

A 2-stage group-sequential adaptive design with O'Brien and Fleming shaped boundaries was applied for this study. An interim analysis was performed to investigate on futility, safety and efficacy of AP-325 or placebo and to do a sample size re-estimation. The analysis was done when the 5-day average pain intensity score as measured at Day 10 had been evaluated by 50% of the subjects (i.e. 48 subjects), data required for this endpoint had been entered into the eCRF, and those data had been cleaned appropriately. A data snapshot was done for the interim analysis.

The investigation on futility, safety and efficacy and the sample size re-estimation was done by an independent data monitoring committee (IDMC) consisting of independent clinical and statistical experts. Only the IDMC and an external statistical advisor as specified in an IDMC charter for supporting the sample size re-estimation had access to unblinded data. The recommendation based on the investigation on futility, safety and efficacy (the recommendation whether study should be stopped or should be continued) and the results of the sample size re-estimation (number of subjects to be screened) was provided to the sponsor.

### Safety and Adverse Event Analysis

Safety analyses were performed using standard descriptive methods and Adverse Events (AEs) were coded by using the medical dictionary for regulatory activities (MedDRA).

An overview table was prepared showing the number of subjects with at least 1 Adverse Event (AE), 1 Treatment Emerged Adverse Event (TEAE), related TEAE, serious TEAE, non-serious TEAE, SAE, related serious TEAE, TEAE leading to withdrawal of study medication, and AE leading to death as well as the total number of events in each category.

Additionally, TEAEs were tabulated by system organ class and preferred term.

Summary tables were prepared for all TEAEs, serious TEAEs, non-serious TEAEs, and TEAEs leading to withdrawal as well as for TEAEs/serious TEAEs sorted by relationship and severity. Summary tables were prepared including counts and percentages of subjects who experienced at least 1 TEAE and the total number of events for that TEAE.

### Ethical and Legal Requirements

The study was performed in accordance with the International Council for Harmonization (ICH) guideline for good clinical practice (GCP; CPMP/ICH/135/95), the appropriate national regulations and the Declaration of Helsinki in its currently acknowledged version.

Approval for this protocol was obtained from national regulations and the Independent Ethics Committee (IEC).

### Results and Discussion

### The Pain Intensity Numeric Rating Scale (PI-NRS)

The pain intensity numeric rating scale (PI-NRS) includes an 11-point scale for the subject self-reporting of pain. The scale ranges from 0 to 10, where 0 is no pain and 10 is the worst pain imaginable. The subjects have kept a diary from Day −7 until the end of the study, in which they recorded their daily pain intensity using a 11-point PI-NRS.

For the analysis of the study endpoints, the daily scores on the PI-NRS is averaged over 5 days (5-day average score) except for Day 5 where the PI-NRS is averaged over 4 days (4-day average score). The averages are rounded to two decimal places.

The 5-day average scores have been calculated for the time points Day 10, 15, 20, 25, 30, and 35 by averaging the scores from the previous 5 days, where at least 4 assessments must be available. The percentage change from baseline is calculated as follows: PI-NRS change=([4/5-day average on Day 5, 10, 15, 25 or 35/5-day baseline average]×100)−100. This means a negative percentage change from Baseline corresponds to an improvement of the PI-NRS.

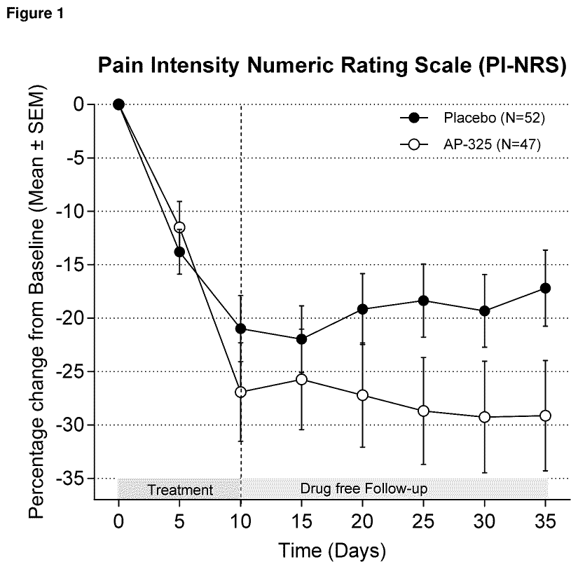

In FIG. 1 the mean percentage change in the 5-day average PI-NRS scores for Day 5, 10, 15, 20, 25, 30 and 35 are displayed with the standard error of the mean as error bars. FIG. 1 shows the change from baseline to Day 10. AP-325 treated patients show a greater change from baseline when compared to placebo treated patients. To assess the difference in the PI-NRS daily pain score between treatments over time (longitudinal analysis) a linear mixed-effects model is used.

The Responder Rate Derived from PI-NRS Score Reduction

The responder rate is derived by calculating the PI-NRS score reduction:

PI-NRS score reduction=100−([4/5-day average on Day 5, 10, 15, 25 or 35/5-day baseline average]×100)
30% Responder=subjects who have a ≥30% reduction in the 5-day average PI-NRS score relative to Baseline.
50% Responder=subjects who have a ≥50% reduction in the 5-day average PI-NRS score relative to Baseline.
70% Responder=subjects who have a ≥70% reduction in the 5-day average PI-NRS score relative to Baseline

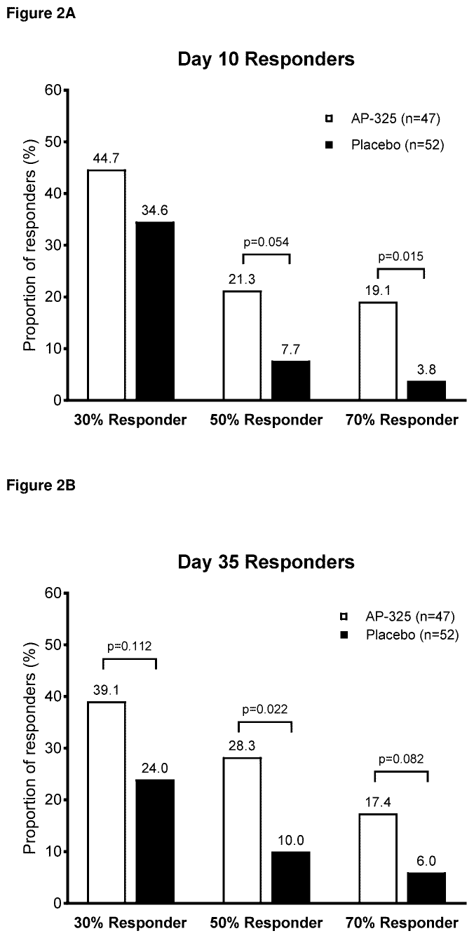

The number and percentage of responders (30%, 50% and 70% reduction) are shown by day and treatment group. The percentage of responders are illustrated by day and treatment group using a bar plot, Day 10 is shown in FIG. 2A and Day 35 is shown in FIG. 2B.

The responder (30%, 50% and 70% reduction) rate (binary variable on patient level) are compared between treatments on Day 10, 15, 20, 25, 30 and 35 (Table 1, 2 and 3). The Day 10 (FIG. 2A) and Day 35 (FIG. 2B) comparison revealed that the responder rate of patients with a 30%, 50% and 70% reduction is higher in AP-325 treated patients when compared to placebo treated patients, these differences are statistically significant for the 50% and 70% responder on Days 10 and 35.

TABLE 1
Success defined as 30% decrease or more

| Time point | n(%) success | n(%) success | | |
|---|---|---|---|---|
| (days) | AP-325 | Placebo | OR (95% CI) | p-value |
| 5 | 6 (12.8%) | 8 (15.4%) | 0.8 (0.3 to 2.5) | 0.712 |
| 10 | 21 (44.7%) | 18 (34.6%) | 1.5 (0.7 to 3.5) | 0.311 |
| 15 | 18 (38.3%) | 19 (36.5%) | 1.1 (0.5 to 2.4) | 0.858 |
| 20 | 18 (39.1%) | 14 (27.5%) | 1.7 (0.7 to 4.0) | 0.226 |
| 25 | 17 (37.0%) | 15 (29.4%) | 1.4 (0.6 to 3.3) | 0.435 |
| 30 | 18 (39.1%) | 13 (25.5%) | 1.9 (0.8 to 4.5) | 0.153 |
| 35 | 18 (39.1%) | 12 (24.0%) | 2.0 (0.9 to 5.0) | 0.112 |

TABLE 2
Success defined as 50% decrease or more

| Time point | n(%) success | n(%) success | | |
|---|---|---|---|---|
| (days) | AP-325 | Placebo | OR (95% CI) | p-value |
| 5 | 1 (2.1%) | 1 (1.9%) | 1.1 (0.0 to 28.6) | 0.943 |
| 10 | 10 (21.3%) | 4 (7.7%) | 3.2 (1.0 to 12.6) | 0.054 |
| 15 | 10 (21.3%) | 6 (11.5%) | 2.1 (0.7 to 6.6) | 0.192 |
| 20 | 13 (28.3%) | 6 (11.8%) | 3.0 (1.0 to 9.2) | 0.041 |
| 25 | 13 (28.3%) | 6 (11.8%) | 3.0 (1.0 to 9.2) | 0.041 |
| 30 | 13 (28.3%) | 5 (9.8%) | 3.6 (1.2 to 12.2) | 0.019 |
| 35 | 13 (28.3%) | 5 (10.0%) | 3.6 (1.2 to 12.0) | 0.022 |

TABLE 3
Success defined as 70% decrease or more

| Time point | n(%) success | n(%) success | | |
|---|---|---|---|---|
| (days) | AP-325 | Placebo | OR (95% CI) | p-value |
| 10 | 9 (19.1%) | 2 (3.8%) | 5.5 (1.4 to 40.4) | 0.015 |
| 15 | 7 (14.9%) | 2 (3.8%) | 4.4 (1.0 to 30.5) | 0.057 |
| 20 | 7 (15.2%) | 2 (3.9%) | 4.4 (1.0 to 30.7) | 0.056 |
| 25 | 7 (15.2%) | 3 (5.9%) | 2.9 (0.8 to 14.0) | 0.134 |
| 30 | 8 (17.4%) | 3 (5.9%) | 3.4 (0.9 to 16.4) | 0.076 |
| 35 | 8 (17.4%) | 3 (6.0%) | 3.3 (0.9 to 15.9) | 0.082 |

Generally speaking, the odds ratio (OR) is a measure of the strength of association with an exposure and an outcome. An OR>1 means greater odds of association with the exposure and outcome. An OR=1 means there is no association between exposure and outcome. An OR<1 means there is a lower odds of association between the exposure and outcome. In the present context of comparing two groups such a first group of patients that are being treated with AP-325 and a second group of patients that are being treated with a placebo, an odds ratio of 1 means that there no difference between the odds in both groups, while an odds ratio of >1 means that the odds of the first group are higher and an odds ratio of <1 means, that the odds of the first group are lower. An OR>1.8 is clinically relevant, meaning a compound of interest shows the desired therapeutic efficacy. Using here, as an illustrative example Table 3 wherein the treatment success is defined as 70% or more, it is shown at Day 10 that the odds ratio is 5.5. This means, the chance of having a treatment success of at least 70% pain reduction is about 5.5 times higher for the AP-325 treated patients than it is for the placebo treated patients (Table 1, 2 and 3).

### Secondary Data Analysis: Post-Hoc Analyses of Group-Mean PI-NRS Scores

Secondary Data analysis by Post-hoc analyses estimated group-mean PI-NRS scores over the follow-up time. It was decided to use a more efficient to detect differences between treatment groups. The purpose of this analysis is to make an estimate based on a model that uses all measurements over time instead of a direct estimate of the difference on day 10 by comparing the measurement at that time. By doing so there is more precision and the assumption is made that pain decreases gradually over time.

Raw PI-NRS data were transposed from wide-format to long-format. Subsequently, linear mixed effects regression was used to estimate the difference between AP-325 and placebo at day 10 after starting treatment, and the interaction between treatment allocation and time. The regression model contained three levels: observations (1) clustered within patients (2) clustered within countries (3). The correlation of observations over time was estimated using an autoregressive model of the first order (AR1). Results are expressed as regression coefficients including 95% confidence intervals and the associated p-value. In total, 99 participants contributed PI-NRS data, of whom 47 (47.5%) were randomly allocated to the AP-325 group. Over all days combined, participants contributed 4119 PI-NRS measurements of which 3430 (83.3%) were measurements after treatment started. Based on the regression model, the estimated mean difference between AP-325 and placebo was 0.88 points on the PI-NRS in favor of AP-325, 95% Cl: 0.22 to 1.54, p=0.009.

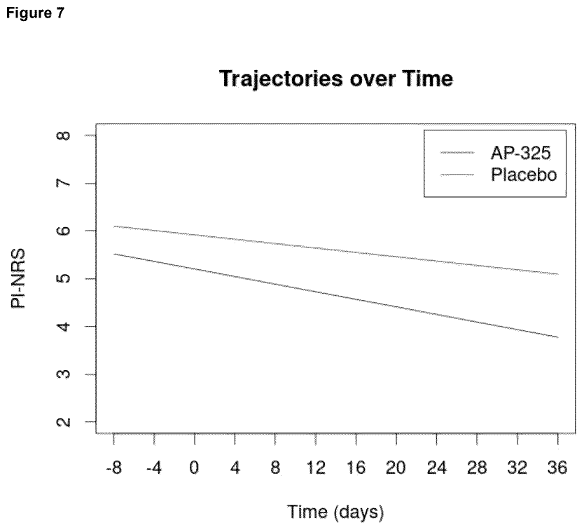

The interaction between treatment allocation and time was statistically significant, indicating that the PI-NRS trajectories over time had a different slope for both groups. Based on the interaction, the regression coefficient for AP-325 was larger, indicating a steeper decrease in PI-NRS over time (see FIG. 7).

### Time to First Use of Rescue Medication

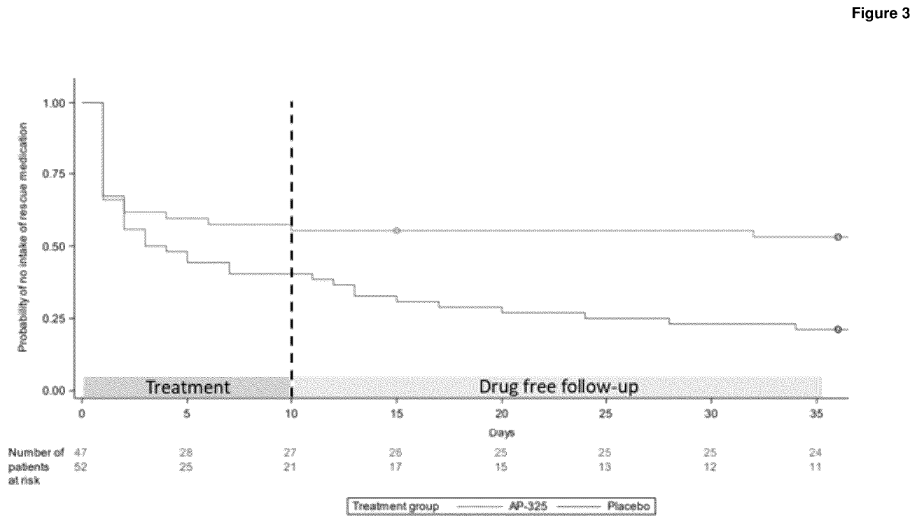

The time to first use of rescue medication after randomization was analysed using Kaplan-Meier methods and a log-rank test. The time to first rescue medication is presented by treatment group in FIG. 3. Also shown is the number of patients with events (i.e. intake of rescue medication) and censored (e.g. premature withdrawal). The time-to-first use is illustrated using Kaplan-Meier plots (FIG. 3).

The time to first rescue medication is derived by date of first use of rescue medication at/after randomization minus date of randomization+1. This means that if a patient takes rescue medication (acetaminophen) at time of randomization, the time to first use of rescue medication will be 1 day. If a patient takes no rescue medication at/after randomization, the patient will be censored on the date of study termination/completion. FIG. 3 shows that there are only 20 AP-325 treated patients that took rescue medication during the first 10 days after randomization (treatment period) compared to 31 placebo treated patients. During the drug free follow-up period (Day 11 till Day 35) the number of placebo treated patients that start taking rescue medication strongly increased, at the end of study (Day 35), 41 placebo treated patients (79%) took rescue medication during the course of the study. In the AP-325 treated group there were only 2 patients that start to take rescue medication during the drug free follow-up period and 1 patient was censored (premature withdrawal). At the end of study only 23 patients from the AP-325 treatment group took rescue medication (49%), the difference between AP-325 treated patients and placebo treated patients on the time to first use of rescue medication is statistically highly significant.

### Daily Sleep Interference

The Daily Sleep Interference Scale (DSIS) was developed to quantify sleep interference due to pain (Vernon M K, 2008). The Daily Sleep Interference Scale (DSIS) consists of an 11-point Likert scale with which patients assess how pain has interfered with their sleep during the past 24 hours. On this scale 0 indicates “pain does not interfere with sleep” and 10 indicates “pain completely interferes with sleep”. Self-assessment was performed daily at awakening.

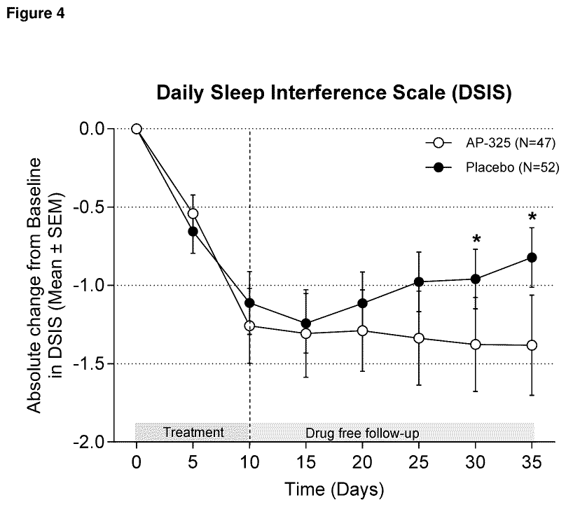

The patients assessed their sleep of the past night daily in the morning in a diary using the DSIS from Day −7 to Day 36. For the analysis of the study endpoints, the daily scores on the DSIS is averaged over 5 days (5-day average score) except for Day 5 where the DSIS is averaged over 4 days (4-day average score). The averages are rounded to two decimal places. FIG. 4 illustrates the mean change in the 5-day average DSIS scores for Day 5, 10, 15, 20, 25, and 35 in a line graph including standard error of the mean as error bars.

Differences in change from Baseline to Day 5, Day 10, Day 15, Day 20, Day 25 and Day 35 in the 5-day average DSIS are compared between treatments using linear regression. The analysis revealed that over time the differences between AP-325 and placebo treated patients was statistically significant on Day 30 and 35. Thus, AP-325 treated patients sleep better with less interferes due to pain during the night (FIG. 4).

### Hospital Anxiety and Depression Scale (HADS)

The Hospital Anxiety and Depression Scale (HADS) is a self-report questionnaire that is used to assess anxiety and depression in hospital patients. It is a brief and easy-to-administer tool that can be used to quickly and accurately identify patients who may be experiencing these mental health conditions. It consists of 14 items, with 7 items on the subscale anxiety and 7 items on the subscale depression. Each item ranges from 0 (best) to 3 (worst). The score range for each subscale is 0-21 and the total score range is 0-42.

Anxiety and depression are common comorbidities in patients with neuropathic pain. Higher HADS scores for both anxiety and depression are significantly associated with higher pain intensity in neuropathic pain conditions. The HADS provides valuable information beyond just pain intensity, allowing for a more comprehensive evaluation of treatment efficacy.

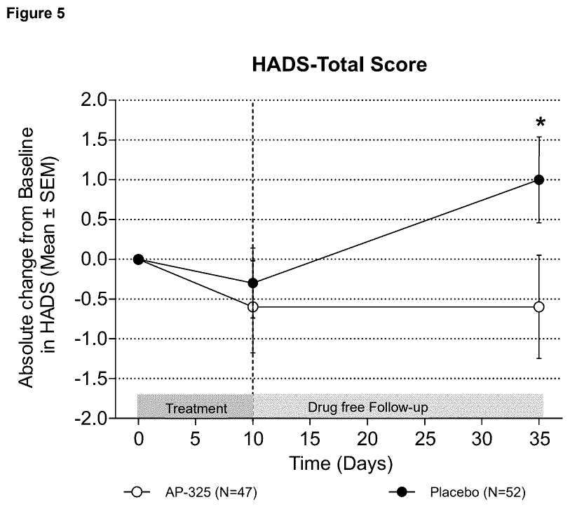

Differences in change from Baseline to Day 10 and Day 35 in HADS total score are compared between treatments using linear regression (FIG. 5). On Day 35 the difference between the AP-325 treated patients and the placebo treated patients on the HADS-Total Score was statistically significant. AP-325 treated patients reported less anxiety and depressive symptoms based on HADS scores.

### Patient Global Impression of Change (PGIC)

The Patient Global Impression of Change (PGIC) is a patient-reported outcome measure used in clinical research to assess a patient's perception of their overall change in health status over time. The subjects will be asked to choose 1 of 7 options (by ticking a box) with option 1 being ‘very much improved’ and option 7 being ‘very much worse’.

Table 4. Copy of the Patient Global impression of Change (PGIC) questionnaire

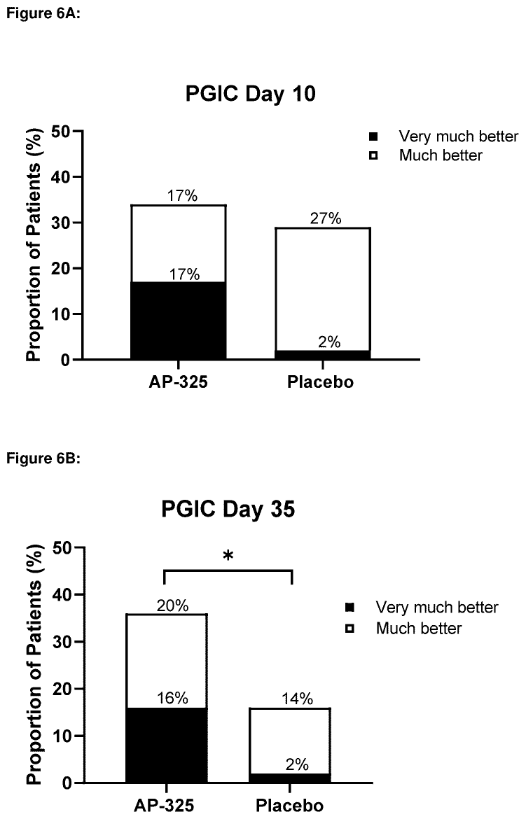

The PGIC is dichotomized into treatment success (i.e. scoring ‘much improved’ or ‘very much improved’) relative to Baseline at Day 3, Day 10, Day 15, and Day 35. The PGIC rating as well as the dichotomized treatment success are displayed by treatment group. A bar plot with the percentage of patients with treatment success for Day 10 (FIG. 6A) and Day 35 (FIG. 6B) are shown.

The hypothesis of a difference in proportions of subjects reporting treatment success based on the PGIC between treatments on 10 and Day 35 was considered using the Pearson's chi-square test. FIG. 6A shows that the proportion of patients that reports a treatment success on Day 10 is comparable between AP-325 treated patients and placebo treated patients. However, the proportion of patients that report “very much better” is 16% after AP-325 treatment and only 2% after placebo treatment. At the end of study on Day 35, it is shown (FIG. 6B) that there is a statistically significant difference between AP-325 treated patients and placebo treated patients in the proportion of patients that reports a treatment success.

Despite a drug free follow-up period of 26 days there are still 36% of the AP-325 treated patients that report treatment success compared to 16% of the placebo treated patients, this difference is statistically significant.

TABLE 5
Treatment Emergent AE (TEAE) in ≥2 Patients

| | Placebo N = 55 | AP-325 N = 48 |
|---|---|---|
| | n (%) | n (%) |
| Subjects with any TEAEs | 31 (56.4) | 23 (47.9) |
| Diarrhoea | 5 (9.1) | 2 (4.2) |
| Flatulence | 2 (3.6) | 1 (2.1) |
| Nausea | 2 (3.6) | 1 (2.1) |
| Asthenia (lack of energy) | 0 | 2 (4.2) |
| Fatigue | 2 (3.6) | 0 |
| Nasopharyngitis | 2 (3.6) | 1 (2.1) |
| Tonsillitis | 0 | 2 (4.2) |
| Blood creatine kinase increased | 1 (1.8) | 3 (6.3) |
| Blood triglycerides increased | 2 (3.6) | 1 (2.1) |
| Back pain | 1 (1.8) | 2 (4.2) |
| Headache | 12 (21.8) | 8 (16.7) |
| Migraine | 2 (3.6) | 0 |

The safety profile of the investigational product appears to be favorable, with TEAEs observed in the active treatment group being comparable to those in the placebo group (Table 5). Both groups exhibited generally mild adverse events, with no significant differences in frequency or severity. Notably, no Serious Adverse Events (SAEs) were reported, and safety-related parameters remained within normal limits throughout the study.

Of particular interest is the absence of central nervous system-related TEAEs in the active treatment group. This finding is especially significant as it suggests a lack of potential for addiction or abuse, which is a critical consideration in drug development and regulatory approval processes. The clean side effect profile, closely mirroring that of placebo, further supports the promising safety profile of AP-325.

### The Number Needed to Treat (NNT)

The Number Needed to Treat (NNT) for 50% pain relief is a measure used to quantify the effectiveness of pain management treatments. It represents the average number of patients who need to receive a specific treatment for one additional patient to achieve at least a 50% reduction in pain intensity compared to a control group or alternative treatment.

AP-325 demonstrates a remarkably low Number Needed to Treat (NNT) of 3.5 for neuropathic pain, positioning it as a potentially highly effective treatment option. This NNT is significantly lower than several first- and second-line pharmacotherapies currently used in neuropathic pain management (Table 6).

TABLE 6
Pharmacotherapy for neuropathic pain in adults

| | | |
|---|---|---|
| Treatment | Number | |
| For neuropathic | Needed to Treat | Adverse drug effects/ |
| pain | (NNT) | particularities |

Recommended first-line

| | | |
|---|---|---|
| Anticonvulstants* | NNT = 8.7 | CNS Symptoms e.g. Feeling tired |
| (Pregabalin, | | or drowsy, dizziness, nausea |
| Gabapentin | | |
| Serotonin | NNT = 7.4 | Feeling tired or drowsy, dizziness, |
| noradrenaline | | nausea, dry mouth, headache |
| reuptake | | |
| inhibitors | | |
| (Duloxetin) | | |
| Tricyclic | NNT = 4.6 | Dizziness, drowsiness, fatigue, |
| Antidepressants | | weight gain |
| (Amitriptylin | | |

Recommended second-line

| | | |
|---|---|---|
| Capsaicin 8% | NNT = 13.2 | Skin reactions/application by |
| patches | | doctors or medical professionals |
| | | supervised by a doctor; patch can |
| | | be cut to size |
| Lidocaine 5% | NNT = 14.5 | Skin reactions/patch can be cut to |
| plasters | | size; restricted authorization: post- |
| | | zoster neuralgia |

Recommended third-line

| | | |
|---|---|---|
| Opioids | NNT = 5.9 | Feeling sleepy (“spaced-out”), |
| | | nausea, vomiting, constipation, |
| | | severe itching, high likely hood of |
| | | dependence/addiction |

Present

| | | |
|---|---|---|
| AP-325 | NNT = 5.8 | No CNS symptoms, clean side |
| | | effect profile |

Reproduced from: Pharmacotherapy and non-invasive neuromodulation for neuropathic pain: a systematic review and meta-analysis, Nadia Soliman*, Xavier Moisset*, Michael C Ferraro, Daniel Ciampi de Andrade, Ralf Baron, Joletta Belton†, David L H Bennett, Margarita Calvo, Patrick Dougherty, Ian Gilron, Aki J Hietaharju, Koichi Hosomi, Peter R Kamerman, Harriet Kemp, Elena K Enax-Krumova, Ewan McNicol, Theodore J Price, Srinivasa N Raja, Andrew S C Rice, Blair H Smith, Fiona Talkington†, Andrea Truini, Jan Vollert, Nadine Attal*, Nanna B Finnerup*, Simon Haroutounian*, NeuPSIG Review Update Study Group. Lancet Neurol 2025; 24: 413-28; and Ahmadi R, Kuner R, Weidner N, Keßler J, Bendszus M, Krieg S M: The diagnosis and treatment of neuropathic pain, Deutsches Ärztblatt International 2024; 121: 825-32. DOI: 10.3238/arztebl.m2024.0215.

The AP-325 combination of high efficacy (low NNT) and a favorable safety profile addresses a significant unmet need in neuropathic pain management. This unique profile could potentially offer patients a more effective treatment option with fewer side effects, potentially improving both pain control and quality of life for those suffering from neuropathic pain.

### Followup Clinical Trials

The following considerations are planned in clinical trials taking the above results into consideration.

### Follow Up Trial Study Objectives

### Follow Up Trial Primary Objectives:

### Objective 1—Dose-Finding at Week 12

To determine the optimal dose of AP-325 by evaluating the efficacy and safety after 12 weeks of once-daily treatment in adults with Peripheral postsurgical neuropathic pain. Where optimal dose is defined as the dose demonstrating the most favourable balance of clinical efficacy and acceptable safety/tolerability.

Objective 2—Maintenance of Effect from Week 13-24

To determine whether the therapeutic effect expected at Week 12 is sustained through Week 24, 12 weeks after cessation of study medication, by comparing the Change From Baseline in the Weekly Average of Daily Pain Intensity (last 7-day mean of past 24-hour average pain scores) on the numeric rating scale (NRS).

### Follow Up Trial Secondary Objectives:

To investigate the safety and tolerability of each dose level of AP-325 in subjects with painful PPNP

To evaluate the full pharmacokinetic profile of each dose level of AP-325

To evaluate pharmacokinetic-pharmacodynamic relationships, if data permit

Follow up trial study design This is a Phase IIb Double Blind, Dose-ranging, placebo controlled, parallel group study to evaluate the efficacy and safety of AP-325 in subjects with peripheral postsurgical neuropathic pain.

### Follow Up Trial Screening Period

Subjects will undertake a screening period, which will be within 21 days of the baseline period (Days −35 to −14).

### Follow Up Trial Run-In Period

Subjects who meet eligibility criteria will enter the run-in period of 2 weeks (Days −14 to −1).

Follow up trial wash-out: During the first week of the run-in period (Days −14 to −8) subjects will be required to wash-out prohibited medications, including all medications used for neuropathic pain. Rescue medication will be provided (paracetamol up to 3000 mg per day), the use of rescue medication must be documented. Only subjects who continue to meet pain intensity criteria at the end of the run-in period will be randomized. Subjects will record their daily pain intensity using the 11-point pain intensity numeric rating scale (PI-NRS) using a diary.

Follow up trial baseline: The weekly average of the PI-NRS collected on the 7 days prior to randomization (Days −7 to −1, minimum 5 entries) will be defined as the baseline. Subjects with at least moderate pain intensity (score≥4) will be eligible to continue into the double blind treatment period.

Follow up trial placebo response mitigation: The run-in period will be used to incorporate 2 methods to mitigate the placebo response:
1. The Placebell©™ Covariate approach will be incorporated; this approach is intended to robustly predict the range of placebo responsiveness in clinical trial participants with minimal trial burden and no additional study risk.
2. Placebo Response Reduction training programs for study staff will be included

### Follow Up Trial Double Blind and Drug Free Period

Approximately 355 eligible subjects will be randomized in a 1:1:1 ratio to AP-325 50 mg, AP-325 100 mg or placebo and enter the double blind period of the study on Day 1. They will then commence with 12 weeks of double blind randomized treatment (Days 1-84) followed by 84 days drug free period (Days 85-168) and continue to document their daily pain scores using the PI-NRS in the diary. End of Study Visit (ESV) will be on Day 168.

Subjects will visit the clinic at screening, at the start of run-in (Day −14), after the wash-out of prohibited medication (Day −8), at randomization (Day 1), at steady state (Day 7), 4 week treatment (Day 28), 8 week treatment (Day 56), at the end of the treatment period (Day 84), Day 112 and 12 week drug-free follow-up period visit (Day 168). Efficacy and safety assessments will be performed as detailed in the Schedule of Activities.

Follow up trial treatment duration and end of study Total study duration will be a maximum of 182 days, after randomization the study will take 168 days

Study residency Study residency is not foreseen, it will be an outpatient study

### Study Endpoints Primary Endpoint:

Follow up trial primary endpoint for objective 1—Dose-Finding at Week 1
Change From Baseline in the Weekly Average of Daily Pain Intensity (last 7-day mean of past 24-hour average pain scores) on the numeric rating scale (NRS), at Week 12 Compared to Placebo
Follow up trial primary endpoint for objective 2—Maintenance of Effect at Week 16, 20 and 24
Change From Baseline in the Weekly Average of Daily Pain Intensity (last 7-day mean of past 24-hour average pain scores) on the numeric rating scale (NRS), at Week 16, 20 and 24 Compared to Placebo

### Follow Up Trial Secondary Endpoints:

Proportion of Participants with ≥30% Reduction From Baseline in the Weekly Average of Daily Pain Intensity on the NRS at Week 12 and Week 16, 20 and 24.
Proportion of Participants with ≥50% Reduction From Baseline in the Weekly Average of Daily Pain Intensity on the NRS at Week 12 and Week 16, 20 and 24.
Proportion of Participants with ≥70% Reduction From Baseline in the Weekly Average of Daily Pain Intensity on the NRS at Week 12 and Week 16, 20 and 24.
Safety and Tolerability as Assessed by Number of Participants With Adverse Events (AEs) and Serious Adverse Events (SAEs) at Week 12 Compared to Placebo.
Proportion of subjects who have “much improved” or “very much improved” relative to baseline on the Patient Global Impression of Change (PGIC) at Week 12 and Week 16, 20 and 24 compared to Placebo.
Change From Baseline in 36-item Short-form Health Status (SF 36v2) Physical Component Summary (PCS) Score at Week 12 and Week 16, 20 and 24 compared to Placebo.
Longitudinal analysis of the difference in the PI-NRS between groups over time from baseline till End of Study.

Follow up trial neuropathic pain evaluation by Neuropathic pain symptom Inventory (NPSI) questionnaire.

Change from Baseline in the Weekly Average of the Daily Sleep Interference Score

Anxiety and depression assessment with the Hospital Anxiety and Depression scale (HADS).

Time to First Rescue Medication after Randomization

Rescue medication use will be monitored daily.

Total amount of rescue medications after randomization Rescue medication use will be monitored daily.

Besides screening and baseline assessments of these parameters, PI-NRS and sleep scale on a daily basis, others as listed in the flow chart.
Follow up trial study treatment Study drug: AP-325
Strength: 50 mg and 100 mg capsules AP-325 orally once daily in the morning (dosing interval is 24 hours+/−4 hours) before meals
One treatment period=84 consecutive days of dosing
Follow up trial control drug: placebo
Placebo capsule once daily in the morning (dosing interval is 24 hours+/−4 hours) before meals
Follow up trial eligibility criteria Inclusion criteria

For inclusion in the study, patients must meet ALL of the following criteria:
1. Patients had to be at least 18 years and not older than 80 years
2. Patients with a diagnosis of Chronic postsurgical Neuropathic pain (Shipton et al., 2008; Schug et al., 2019) defined as chronic neuropathic pain that develops or increases in intensity after a surgical procedure and persists beyond the healing process, i.e., at least 6 months after the surgery. The specific subdiagnoses included are chronic post-surgical neuropathic pain after breast surgery (e.g. breast-conserving surgery, mastectomy, surgery to remove lymph nodes), chest surgery (e.g. thoracotomy, video assisted thoracoscopy and sternotomy), hernia repair of the abdominal wall (e.g. femoral hernia repairs, inguinal hernia repairs, umbilical hernia repair or incisional hernia repair), abdominal surgery (e.g. cholecystectomy, appendectomy), varicose vein surgery or gynecologic surgery (e.g. hysterectomy, C-section).
3. Patients must have probable or definite Neuropathic pain as assessed by the revised NeuPSIG grading system (Finnerup et al., 2016, Haroutiunian et al., 2013)
4. Patients had to be willing and able to discontinue, and washout prohibited substances including:
pain medications (e.g. antidepressants, anticonvulsants/antiepileptics, selective serotonin and dual reuptake inhibitors, opioids, long-acting benzodiazepines, muscle relaxants, and topical analgesics), except the rescue medication, and
for specific washout periods of at least 5 times the drug half-life
Note: Patients using prohibited substances for other indications than neuropathic pain, e.g. antiepileptics for the treatment of epilepsy, could not be included in the study, because a discontinuation of such medication was not medically justifiable.
5. Permitted concomitant medications had to have been stable for at least 4 weeks prior to Day −14 and any non-pharmacological therapies (e.g. physiotherapy, acupuncture and transcutaneous electrical neural stimulation) had to have been initiated at least 3 weeks prior to Screening
6. Female patients had not to be pregnant or breastfeeding and be
a. of non-childbearing potential or
b. if of childbearing potential, had to use a highly effective contraceptive method from start of the IMP intake until 30 days after the last IMP intake and have a negative pregnancy test at Screening (blood test)
7. Male patients had to agree, from start of the IMP intake until 3 months after the last IMP intake, to refrain from donating sperm and use a male condom when having sexual intercourse with a woman of childbearing potential at any time and advise her to use a highly effective contraceptive method
8. Patients had to understand the nature of the study procedures and provide written informed consent prior to any study-related procedures
9. Body weight≥55 kg for men and ≥50 kg for women
10. Body mass index (BMI)<40 kg/m2

### Exclusion Criteria

Patients must not enter the study if ANY of the following exclusion criteria is present:
1. Patients with neuropathic pain not a result of a surgical procedure as defined in inclusion criterion 2
2. Patients with any other coexisting pain that could not be discriminated from post surgical neuropathic pain, in the opinion of the patient or clinician e.g. the pain was at least partially due to pain in deeper structures such as internal organs, joints, muscles or bones
3. Patients using adjuvant chemotherapy or radiotherapy; adjuvant therapies had to have been finished at least 4 weeks prior to the run-in period (Day −14)
4. Inadequate hepatic and renal function at screening defined as:
a. Patients with moderate or severe hepatic impairment as defined by Child-Pugh classification (Class B, or C) will be excluded (See Appendix 1).
b. Patients with moderate or severe renal impairment as defined by the Classification of Renal Function by CKD-EPI eGFR will be excluded (See Appendix 2).
5. Inadequate hematologic function, defined as:
a. Absolute neutrophil count (ANC)<1000 cells/mm3 (1.0×109/L).
b. Platelet count<75,000 cells/mm3 (75×109/L) or <35,000 cells/mm3 (35×109/L) with bone marrow involvement.
6. Uncontrolled hypertension defined as average of triplicate seated SBP>150 mmHg or DBP>90 mmHg at screening after 5 minutes rest.
7. History of severe allergic or anaphylactic reactions to multiple drugs or history of hypersensitivity to active or inactive excipients of the IMP or Rescue Medication (RM).
8. Patients using strong opioids (e.g. a Morphine Equivalent Dose>80 mg/day) 9. Patients taking concomitant CYP2C9 inhibitors and/or inducers.
10. Participants with a positive urine drug screen for illicit substances at screening will be excluded from the study
11. HADS depression subscale score of ≥11
12. Patients with clinically significant GI problems that, in the opinion of the investigator, could interfere with the absorption, metabolism, or safety assessment of the IMP will be excluded from the study
13. Known history of infection with human immunodeficiency virus (HIV).
14. Serologic status reflecting active hepatitis B or C infection.
a. Subjects who test positive for hepatitis B surface antigen (HBsAg) will be excluded from the study. Subjects who are HBsAg negative, regardless of hepatitis B core antibody (anti-HBc) status, can be enrolled.
b. Subjects who test positive for hepatitis C antibody (anti-HCV) will be excluded from the study.
15. Any of the following cardiac criteria:
a. Mean resting corrected QT interval (QTcF) 2_470 msec obtained from 3 electrocardiograms (ECGs).
b. Any clinically important abnormalities in rhythm, conduction or morphology of resting ECG (e.g., complete left bundle branch block, third degree heart block).
c. Any factors that increase the risk of QTc prolongation or risk of arrhythmic events such as heart failure, congenital long QT syndrome, family history of long QT syndrome or unexplained sudden death under 40 years of age. Concomitant medications known to prolong QTc should be excluded
16. Participation of patient in an interventional clinical study within 1 month or, if applicable, 5 half-lives of the IMP, whatever was longer, before Screening or during participation in this study
17. Patients who were previously enrolled in this clinical study and had taken study medication or terminated due to poor compliance
18. Patients dependent (as an employee or relative) on the sponsor or investigator.
19. Judgment by the investigator that the subject should not participate in the study if the subject is unlikely to comply with study procedures, restrictions and requirements.

Sample Size A total of 426 patients completing the study, randomized in a 1:1:1 ratio to AP-325 50 mg (N=142), AP-325 100 mg (N=142) or placebo (N=142) per study arm.

For the sample size calculation, a 2-sided t-test is considered. Assuming a 2-sided significance level of 0.05, a total of 355 subjects is sufficient to reveal a mean difference on the PI-NRS at Week 12 of 0.5 with a standard deviation of 1.5 and a power of 80%. With an assumed replacement rate of 20%, at least 511 subjects should be screened for the study.

Concomitant medication Prohibited medication: All pain medication except rescue medication is prohibited during the study. Antidepressants, anticonvulsants/antiepileptics, opioids, selective serotonin and dual reuptake inhibitors, long-acting benzodiazepines, muscle relaxants, and topical analgesics are not allowed.
Rescue medication: paracetamol 500-1000 mg
Paracetamol 500-1000 mg every 4-6 h if needed but to take no more than 3000 mg in 24 h.
Safety parameters ECG, Vitals: Tbd
Withdrawal questionnaire based on CSS recommendations (20 abuse specific AE's).
PK A full PK assessment will be performed at Day 84 on all subjects.
D14: All subjects Ctrough (aka predose sample)
D28: All subjects Ctrough (aka predose sample)+1 hour after dosing
Urine analysis: renal excretion of AP-325.
CYP Genotyping To examine a potential impact of CYP-2C9 activity on the PK of AP-325, CYP 2C9 genotyping will be performed by using buccal swabs at screening

Biomarker The DHO: the upstream metabolite DHO of the de novo pyrimidine synthesis pathway can be used as a biomarker in blood orand urine to monitor DHODH inhibition in humans (Pontikos et al., 2022). DHO will be measured at screening and baseline (Day 0), at Days 28 the end of treatment (Day 84), and 84 at the end of study (Day 168) in whole blood, samples and urine samples.

Immunophenotyping analysis: Whole blood samples will be collected at baseline (Day 0), at the end of treatment (Day 84), and at the end of study (Day 168) for immunophenotyping analysis. The objective of this assessment is to characterize peripheral immune cell populations and evaluate potential treatment-related changes in immune cell composition.

Samples will be processed for flow cytometry and fluorescence-activated cell sorting (FAGS) analysis. Specific multicolor antibody panels will be used to identify lymphocyte, monocyte, and other relevant leukocyte subsets.

Cytokine analysis: Blood samples will be collected at baseline (Day 0), at the end of treatment (Day 84), and at the end of study (Day 168) and processed to obtain serum or plasma. Samples will be stored at −80° C. until analysis. Cytokine concentrations (e.g., IL-1β, IL-6, TNF-α) will be determined using validated enzyme-linked immunosorbent assay (ELISA) kits or by using multiplex beat based immunoassay specific for human cytokines. All reagents and samples will be prepared according to the manufacturer's instructions.

### Statistics

The primary endpoint, the change from Baseline to Week 12 in the weekly average PI-NRS score in the 100 mg AP-325 group compared to the placebo group will be derived from a linear regression (analysis of covariance [ANCOVA]), corrected for the baseline PI-NRS average pain score and the stratification factor country.

The key secondary endpoint, the 50% responder rate, i.e. the number of patients with an at least 50% decrease in the weekly average PI-NRS score from Baseline to Week 12 will be compared between treatments using the Pearson's chi-square test. The same procedure will be followed for the 30% and 70% responder rates.

The other key secondary endpoint “maintenance of effect” will also use the change from Baseline to Week 16, 20 and 24 in the weekly average PI-NRS score in the 50 mg and 100 mg AP-325 group compared to the placebo group will be derived from a linear regression (analysis of covariance [ANCOVA]), corrected for the baseline PI-NRS average pain score and the stratification factor country.

The PGIC will be dichotomized into treatment success (i.e. scoring ‘much improved’ or ‘very much improved’). The hypothesis of a difference in proportions of patients reporting treatment success on the PGIC between treatments will be considered using Pearson's chi-square test. In case of expected cell counts below 5, the Fisher's exact test will be used instead.

Differences in scores of the NPSI, DSIS, and HADS will be compared between treatments using linear regression (ANCOVA), corrected for the baseline value and country. For the DSIS, weekly averages will be calculated similar to the PI-NRS.

Kaplan-Meier methods and a log-rank test will be used to analyze the time to first classification as treatment failure and the time to first use of rescue medication. The total amount of rescue medication (i.e. the amount of rescue medication per day in mg) and the weekly averages will be tabulated and compared between treatments using a Wilcoxon rank sum test.

To assess the difference in the PI-NRS daily pain score between treatments over time a linear mixed-effects model will be used (longitudinal analysis).

### Missing Data:

To allow intention-to-treat analysis, all missing outcome data will be imputed.

### Child-Pugh Classification System for Hepatic Impairment

TABLE
Child-Pugh Scoring System

| | Points | | |
|---|---|---|---|
| | Assigned | | |
| Parameter | 1 Point | 2 Points | 3 Points |
| Total Bilirubin | <2 mg/dL | 2-3 mg/dL | >3 mg/dL |
| | (<34 μmol/L) | (34-50 μmol/L) | (>50 μmol/L) |
| Serum | >3.5 g/dL | 2.8-3.5 g/dL | <2.8 g/dL |
| Albumin | (>35 g/L) | (28-35 g/L) | (<28 g/L) |
| INR (Prothrombin | <1.7 | 1.7-2.3 | >2.3 |
| Time) | | | |
| Ascites | None | Mild to moderate | Severe (refractory |
| | | (controlled with | to medical |
| | | diuretics) | therapy) |
| Hepatic | None | Grade 1-2 (mild)* | Grade 3-4 |
| Encephalopathy | | | (severe)* |

Total Score Range: 5-15 points

TABLE 2
Child-Pugh Classification

| | | Degree of Hepatic | One-Year | Two-Year |
|---|---|---|---|---|
| Class | Total Score | Impairment | Survival | Survival |
| A | 5-6 points | Mild | 100% | 85% |
| B | 7-9 points | Moderate | 81% | 57% |
| C | 10-15 | Severe | 45% | 35% |
| | points | | | |

### Notes:

Hepatic Encephalopathy Grading (West Haven Criteria):
   Grade 1: Altered mood/behavior, sleep disturbance, mild confusion
   Grade 2: Lethargy, disorientation, inappropriate behavior, asterixis
   Grade 3: Somnolence, severe confusion, incomprehensible speech
   Grade 4: Coma

For patients with Primary Biliary Cholangitis (PBC) or Primary Sclerosing Cholangitis (PSC):
Bilirubin scoring should be modified: 1-4 mg/dL=1 point; 4-10 mg/dL=2 points; >10 mg/dL=3 points

### APPENDIX 2: RENAL FUNCTION CLASSIFICATION BASED ON EGFR

TABLE
Classification of Renal Function by eGFR (FDA Guidance)

| | eGFR | | Study |
|---|---|---|---|
| Category | (mL/min/1.73 m²) | Classification | Eligibility |
| Normal | ≥90 | Normal renal function | Eligible |
| Mild | 60-89 | Mild decrease in renal | Eligible |
| Impairment | | function | |
| Moderate | 30-59 | Moderate decrease in | EXCLUDED |
| Impairment | | renal function | |
| Severe | 15-29 | Severe decrease in renal | EXCLUDED |
| Impairment | | function | |
| End-Stage | <15 (or on | Kidney failure | EXCLUDED |
| Renal | dialysis) | | |
| Disease | | | |
| (ESRD) | | | |

Table: CKD-EPI (2021) Equation for eGFR Calculation

The eGFR will be calculated using the CKD-EPI creatinine equation (2021) without race as a variable:
For females:
If serum creatinine≤0.7 mg/dL: eGFR=142×(SCr/0.7)\hat{ }−0.241×0.9938\hat{ }age
If serum creatinine>0.7 mg/dL: eGFR=142×(SCr/0.7)\hat{ }−1.200×0.9938\hat{ }age
For males:
If serum creatinine≤0.9 mg/dL: eGFR=142×(SCr/0.9)\hat{ }−0.302×0.9938\hat{ }age
If serum creatinine>0.9 mg/dL: eGFR=142×(SCr/0.9)\hat{ }−1.200×0.9938\hat{ }age

### Where:

SCr=serum creatinine in mg/dL
age=patient age in years

### Notes:

eGFR will be calculated at screening using serum creatinine, age, and sex
If screening eGFR is 55-65 mL/min/1.73 m², a repeat measurement may be performed within 7 days to confirm eligibility
Patients on medications known to affect creatinine secretion (e.g., trimethoprim, cimetidine) should have eGFR interpreted with caution

### REFERENCES

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.
Review of neuropathic pain: Nadine Attal, Didier Bouhassira, Lesley Colvin, Advances and challenges in neuropathic pain: a narrative review and future directions, British Journal of Anaesthesia, Volume 131, Issue 1, 2023, Pages 79-92, ISSN 0007-0912, https://doi.org/10.1016/j.bja.2023.04.021.
The Pain Intensity Numeric Rating Scale (PI-NRS) and the Patient Global Impression Of Change (PGIC) scale: Farrar J T, Young J P Jr, LaMoreaux L. Werth J L, Poole M R. Clinical importance of changes in chronic pain intensity measured on an 11 point numerical pain rating scale. Pain. 2001 November; 94(2):149-158.
The Hospital Anxiety and Depression Scale: Zigmond A S, Snaith R P. The hospital anxiety and depression scale, Acta Psychiatr Scand. 1983 June; 67(6):361-70. doi: 10.1111/j.1600-0447.1983.tb09716.x. PMID: 6880820. Licenses for use of the HADS and copies thereof where obtained from the ePROVIDE™ platform by Mapi Research Trust. Mapi Research Trust is a non-profit organization facilitating access to Clinical Outcomes Assessments (COAs) for the entire scientific community and the website for purchasing the HADS questionnaire is available from https:Heprovide.mapi-trust.org/instruments/hospital-anxiety-and-depression-scale.
Schug S A, Lavand'homme P, Barke A, Korwisi B, Rief W, Treede R D et al. The IASP classification of chronic pain for ICD-11: chronic postsurgical or posttraumatic pain. Pain 2019; 160(1):45-52.
Finnerup N B, Haroutounian S, Kamerman P, Baron R, Bennett D L, Bouhassira D, et al. Neuropathic pain: an updated grading system for research and clinical practice. Pain. 2016 August; 157(8):1599-606.
Daily Sleep Interference Scale (DSIS): Vernon M K, Brandenburg N A, Alvir J M, Griesing T, Revicki D A. Reliability, validity, and responsiveness of the daily sleep interference scale among diabetic peripheral neuropathy and postherpetic neuralgia patients. J Pain Symptom Manage. 2008 July; 36(1):54-68. doi: 10.1016/j.jpainsymman.2007.09.016. Epub 2008 Apr. 14. PMID: 18411009.
Axonal degeneration and disruption of axonal transport: Cashman C R, Hoke A. Mechanisms of distal axonal degeneration in peripheral neuropathies. Neurosci Lett. 2015 Jun. 2; 596:33-50. doi: 10.1016/j.neulet.2015.01.048. Epub 2015 Jan. 21. PMID: 25617478; PMCID: PMC4428955.
Neuropathic pain from toxins and DIPN: Peters J, Staff N P. Update on Toxic Neuropathies. Curr Treat Options Neurol. 2022 May; 24(5):203-216. doi: 10.1007/s11940-022-00716-5. Epub 2022 Apr. 6. PMID: 36186669; PMCID: PMC9518699; and Jones M R, Urits I, Wolf J, Corrigan D, Colburn L, Peterson E, Williamson A, Viswanath O. Drug-Induced Peripheral Neuropathy: A Narrative Review. Curr Clin Pharmacol. 2020; 15(1):38-48. doi: 10.2174/1574884714666190121154813. PMID: 30666914; PMCID: PMC7365998. Small fiber neuropathy Hovaguimian A, Gibbons C H. Diagnosis and treatment of pain in small-fiber neuropathy. Curr Pain Headache Rep. 2011 June; 15(3):193-200. doi: 10.1007/s11916-011-0181-7. PMID: 21286866; PMCID: PMC3086960.
Child C G, Turcotte J G. Surgery and portal hypertension. In: The liver and portal hypertension. Edited by CG Child. Philadelphia: Saunders 1964:50-64.
Pugh R N, Murray-Lyon I M, Dawson J L, Pietroni M C, Williams R. Transection of the oesophagus for bleeding oesophageal varices. Br J Surg 1973; 60:646-649.
Inker L A, Eneanya N D, Coresh J, et al. New Creatinine- and Cystatin C-Based Equations to Estimate GFR without Race. N Engl J Med. 2021; 385(19):1737-1749.
FDA Guidance for Industry: Pharmacokinetics in Patients with Impaired Renal Function—Study Design, Data Analysis, and Impact on Dosing and Labeling (2020).
Methods of producing AP-325, including pharmaceutical acceptable compositions thereof are known from WO2012055567A2 and WO2015140081 A1. The journal article J. Med. Chem. 1996, 39, 23, 4608-4621 also provides useful methods and characterization for the synthesis of AP-325 which is also commercially available from several vendors. Useful excipients are also known from US2008/0241070A1.

It will be readily apparent to a person skilled in the art that varying substitutions and modifications may be made to the invention disclosed herein without departing from the scope and spirit of the invention.

As used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group. Further embodiments of the invention will become apparent from the following claims.

1. A method of treating neuropathic pain in human patients comprising
a treatment cycle comprising:
administering a compound according to formula (I), or an isomer thereof, or a tautomer thereof, or an isotope thereof, over a period T¹; formula (I) having the structure of: 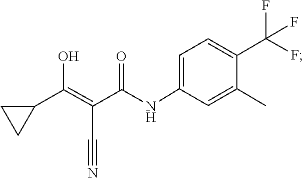 [SMILES: Cc1cc(NC(=O)/C(C#N)=C(\O)C2CC2)ccc1C(F)(F)F]
discontinuing the administration of the compound according to formula (I) for a period T² that immediately follows T¹;
obtaining pain reduction over the total sum of the periods T¹ and T² of the treatment cycle.

2. The method of claim 1, wherein obtaining pain reduction over the total sum of the periods T¹ and T² of the treatment cycle is determined according to pain intensity scores of the Pain Intensity Numeric Rating Scale (PI-NRS).

3. The method of claim 1, wherein for the duration of T², the obtained pain reduction is about equal to or greater than the maximum pain reduction measured during T¹, preferably determined according to pain intensity scores of the Pain Intensity Numeric Rating Scale (PI-NRS).

4. The method of claim 1, wherein for the duration of T², the obtained pain reduction is greater than 15%.

5. The method of claim 1, wherein the absolute pain reduction, over the total sum of the periods T¹ and T² of the treatment cycle is greater than 1.0 point determined relative to a baseline level of pain measured immediately before the period T¹ is begun and measured according to pain intensity scores of the Pain Intensity Numeric Rating Scale (PI-NRS).

6. The method of claim 1, wherein less than 30% of patients undergoing the treatment cycle have had a Treatment Emergent Adverse Event (TEAE) determined at the end of the period of T².

7. The method of claim 1, wherein period T²≥period T¹, preferably period T²≥2×period T¹.

8. The method of claim 1, wherein period T¹ is in the range of from 1 to 100 days.

9. The method of claim 1, wherein period T² is in the range of from 1 to 200 days.

10. The method of claim 1, wherein the patient is dosed daily with a compound according to formula (I) over the period T¹.

11. The method of claim 1, wherein the neuropathic pain is peripheral and/or predominantly peripheral neuropathic pain or central and/or predominantly central neuropathic pain.

12. The method of claim 1, wherein the neuropathic pain is a chronic neuropathic pain.

13. The method of claim 1, wherein none of the patients undergoing treatment experience drug withdrawal, drug withdrawal syndrome, or substance withdrawal syndrome.

14. A method of immune resolution therapy (IRT) comprising
a treatment cycle comprising:
administering a compound according to formula (I), or an isomer thereof, or a tautomer thereof, or an isotope thereof, over a period T¹; formula (I) having the structure of: 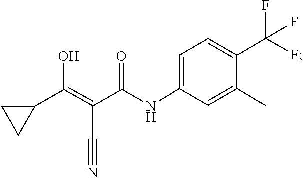 [SMILES: Cc1cc(NC(=O)/C(C#N)=C(\O)C2CC2)ccc1C(F)(F)F]
inhibiting of dihydroorotate dehydrogenase (DHODH) during T¹;
modulating GABA receptors during T¹;
discontinuing the administration of the compound according to formula (I) for a period T² that immediately follows T¹;
clearing the compound according to formula (I) during T²;
modulating lymphocyte subpopulations during T² relative to the last day of T¹, optionally determined by flow cytometry or Fluorescence-Activated Cell Sorting (FACS);
obtaining pain reduction over the total sum of the periods T¹ and T² of the treatment cycle.

15. The method of claim 14, wherein the method of immune resolution therapy (IRT) is for treating neuropathic pain.

16. The method of claim 14, wherein during T² modulating lymphocyte subpopulations comprises increasing the Treg lymphocyte population and the Breg lymphocyte population relative to the Treg lymphocyte population and the Breg lymphocyte population measured at the end of T¹ determined by flow cytometry or FAGS.

17. The method of claim 14, wherein beginning T², the compound according to formula is cleared within 1 to 7 days determined by pharmacokinetic analysis using LC-MS/MS (Liquid Chromatography-Tandem Mass Spectrometry).

18. The method of claim 14, wherein modulating the lymphocyte subpopulation comprises lowering the Th1 lymphocyte population, lowering the Th17 lymphocyte population and lowering the inflammatory B lymphocyte (B cells) population, measured at the end of T¹ relative to the Th1 lymphocyte population, the Th17 lymphocyte population and the inflammatory B lymphocyte (B cells) population measured at the end of T², determined by flow cytometry or FAGS.

19. The method of claim 14, wherein the lymphocyte population measured at the end of T¹ is lower than the lymphocyte population measured as the baseline level immediately before the period T¹ is begun, determined by flow cytometry or FAGS.

20. The method of claim 14, wherein the population of pro-inflammatory CD4+ T cells that secrete IFN-γ and IL-17 measured at the end of T¹ is lower than the population of pro-inflammatory CD4+ T cells that secrete IFN-γ and IL-17 measured as the baseline level immediately before the period T¹ is begun, determined by flow cytometry or FAGS.

21. The method of claim 14, wherein the last day of T², the ratio of the population of regulatory T cells (Tregs) to the population of effector T cells is higher than said ratio measured at the end of T¹ determined by flow cytometry or FAGS.

22. The method of claim 14, wherein the last day of T², the ratio of the population of macrophages in the M2 tissue remodelling, anti-inflammatory phase to the population of macrophages in the M1 pro-inflammatory phase is higher than said ratio measured at the end of T¹ determined by flow cytometry or FAGS.
